(19) United States
(12) Patent Application Publication
(10) Pub. No.: US 2021/0038575 A1
(43) Pub. Date: Feb. 11, 2021

(54) USE OF 1-PHENYL-2-PYRIDINYL ALKYL ALCOHOL DERIVATIVES FOR TREATING CYSTIC FIBROSIS

(71) Applicant: CHIESI FARMACEUTICI S.p.A., Parma (IT)

(72) Inventors: Gino VILLETTI, Parma (IT); Serena BERTOLINI, Parma (IT); Claudio SORIO, Parma (IT)

(73) Assignee: CHIESI FARMACEUTICI S.p.A., Parma (IT)

(21) Appl. No.: 16/769,074

(22) PCT Filed: Dec. 19, 2018
(86) PCT No.: PCT/EP2018/085965

§ 371 Date: Jun. 2, 2020

(30) Foreign Application Priority Data
Dec. 28, 2017 (EP) 17210827.6

(51) Int. Cl. A61K 31/44; A61K 9/00; A61K 31/47; A61K 31/443

(52) U.S. Cl. CPC A61K 31/44; A61K 31/443; A61K 31/47; A61K 9/0078

(57) ABSTRACT

The present invention relates to the use of agents, which are 1-phenyl-2-pyridinyl alkyl alcohol derivatives, for the prevention and/or treatment of cystic fibrosis in a subject, wherein the subject is characterized by at least one mutation in the gene encoding the CFTR protein, wherein the at least one mutation is causative for incorrect folding and/or processing of the CFTR protein. By the use of the compound according to the present invention, cystic fibrosis in the subject may be prevented or treated. The agent to be used according to the present invention has the capacity to restore the presence of the mutant CFTR protein at the cell surface, and thus act as CFTR correctors. The agent to be used according to the present invention may be administered to a subject in need thereof alone or in combination therapy with other agents, and is suitably administered by inhalation.

### INTRODUCTION

### Field of the Invention

The present invention relates to the use of specific 1-phenyl-2-pyridinyl alkyl alcohol derivatives in the treatment of cystic fibrosis.

### BACKGROUND OF THE INVENTION

Cystic fibrosis (CF) is a common lethal genetic disease caused by mutations of the gene coding for the cystic fibrosis transmembrane regulator (CFTR), a chloride channel. The disease is a multisystem disease characterized by pancreatic insufficiency and chronic airway infections, decreased lung function, repeated pulmonary exacerbations and respiratory failure. The disease is autosomal recessive and is caused by decreased levels and/or deficient activity of the CFTR channel, an ABC transporter for anions that is normally present on the apical surface in the epithelial membrane of many cells, including airway cells (Leier et al., 2012, Cell Physiol. Biochem., vol. 29, p. 775-790; Wainwright et al., 2015, N. Engl. J. Med., vol. 373, p. 220-231; Lambert et al., Am. J. Respir. Cell Mol. Biol., 2014, vol. 50, p. 549-558). The abnormal salt and water transport at epithelial cell surfaces caused by mutation of CFTR leads inter alia to exaggerated mucus secretion, and infection or inflammation in affected organs. No fully satisfying medical treatment for cystic fibrosis is available to date.

Although many mutations in the CFTR protein have been described to be causative for cystic fibrosis in humans, a mutation causing deletion of phenylalanine 508 (Phe508del; F508del; ΔF508) is the most common mutation in the CFTR gene; about 90% of cystic fibrosis patients are heterozygous for a respective mutation, and almost half of the patients are homozygous for this mutation (Kuk et al., 2015, Ther. Adv. Resp. Dis., vol. 9, p. 313-326). The mutation ΔF508 impairs the folding/triggers misfolding of the CFTR protein, leading to premature degradation of the translated protein, and is thus causative for a defect that severely reduces protein levels at the epithelial membrane (Blanchard et al., 2014, FASEB J., vol. 28, p. 791-801); in addition, the mutation ΔF508 impairs CFTR protein stability and gating: the few channels that are present at the cell surface have limited chloride/bicarbonate ion transport activity and are thus functionally impaired (Leier et al., supra; Wainwright et al., supra). In contrast, other CFTR mutants have been described wherein the CFTR protein is properly present at the epithelial cell surface but is characterized by a deficiency in its chloride/bicarbonate gating/conductance, for example the CFTR mutant G551D (Kuk et al., supra). The “Cystic Fibrosis Mutation Database”, accessible online at http://www.genet.sickkids.on.ca/app is a comprehensive database providing an overview of known CFTR mutations implicated in cystic fibrosis. According to said database, there are currently more than 2000 CFTR mutations known, grouped in missense mutations, frameshift mutations, splicing mutations, nonsense mutations, in frame insertions/deletions (in/dels), large in/dels, promoter mutations, sequence variations and mutations of still unknown molecular effect. The known mutations are found distributed throughout the open reading frame of the CFTR gene (including 27 exons and 26 introns), as well as the promotor and the 3′ untranslated region (3′ UTR) of the CFTR gene. “Sequence variation” refers to all those genetic mutations which are as such known, but which have not been shown to be disease causing; however, when a sequence variation is found in one single individual, it is not possible to determine if it is “not disease causing”. A human sequence variation which has been shown to be not disease causing and which is present in an allelic frequency of 1% is also termed “polymorphism”, see http://www.genet.sickkids.on.ca/app.

Mucus Production, Inflammation and cAMP Levels in Cystic Fibrosis

Cystic fibrosis is typically also characterized by overproduction of mucus, i.e. a viscoelastic biological material that is a composite of components secreted apically (luminally) by epithelial and glandular cells and covers and protects the apical surfaces of the respiratory, gastrointestinal, and reproductive epithelial tracts. Overproduction of mucin glycoproteins (“mucins”) and mucus plugging is usually most fatal in the airways of cystic fibrosis patients. However, the mucus overproduction is presently not understood to be a direct cause of a defective CFTR protein but, rather, to be a downstream consequence; in the lungs, the expression of mucin genes was shown to be triggered by inflammation resulting from chronic infection (Kreda et al., Cold Spring Harb. Perspect. Med., 2012, a009589). Inflammation, in turn, is fomented by decreased levels of cyclic adenosine monophosphate (cAMP). As is commonly known, cAMP is a ‘second messenger’ molecule that is generated by the enzyme adenyl cyclase and is involved in regulation of a variety of cellular processes including airway smooth muscle relaxation and inflammatory mediator release. In the body, cAMP is hydrolyzed by specific enzymes of the phosphodiesterase (PDE) family, and thus, activation of adenyl cyclase and/or inhibition of specific PDE enzymes represents a potential mechanism by which cell functions including airway smooth muscle relaxation and release of inflammatory mediators may be regulated. Eleven PDE gene families (PDE1-11) have been identified. Among these, PDE4, which hydrolyzes cAMP, is a well-studied enzyme expressed in many inflammatory and immunomodulatory cells. The PDE4 gene family is comprised of four genes (PDE4A, B, C, D), each with several splice variants, and PDE4 expression has a broad tissue distribution, including brain, gastrointestinal tract, spleen, lung, heart, testis, kidney, and almost all inflammatory cell types (Abbott-Banner et al., 2014, Basic Clin. Pharmacol. Toxicol., vol. 114, p. 365-376). In the lungs cAMP is involved in the regulation of many functions related to inflammatory cells, mucociliary clearance, and fibrotic and pulmonary vascular remodeling. In particular, high cAMP levels stall the activity of immune and inflammatory cells, such as neutrophils, T-lymphocytes and macrophages (Soto et al., Curr. Opin. Pulm. Med., 2005, vol. 11, p. 129-134).

Thus, it has been proposed that a cAMP elevating agent, such as a PDE4 inhibitor, would be useful in the treatment of respiratory diseases associated with mucus overproduction, such as COPD and bronchitis (Page et al., Curr. Opin. Pharmacol., 2012, vol. 12, p. 275-286), and possibly cystic fibrosis. Indeed, some PDE4 inhibitors were demonstrated to inhibit inflammatory cytokine and mediator release from inflammatory cells, inhibit migratory activity of these cells and can even promote their apoptosis (Kawamatawong, J. Thorac. Dis., 2017, vol. 9, p. 1144-1154). Roflumilast (3-cyclo-propylmethoxy-4-difluoromethoxy-N-[3,5-di-chloropyrid-4-yl]-benzamide; Hatzelmann et al., 2001, Pharmacol. Exp. Ther. Vol. 297, p. 267-279) is a PDE4 inhibitor which has been clinically approved for use in COPD patients with chronic bronchitis (e.g. Beghè et al., Am. J. Respir. Crit. Care Med., 2013, vol. 188, p. 271-278). Alternative PDE4 inhibitors proposed for treatment of diseases of the respiratory tract characterized by airway obstruction include 1-phenyl-2-pyridinyl alkylene alcohols and derivatives thereof (WO 2008/006509 A1, WO 2009/018909 A2 and WO 2010/089107 A1).

cAMP-stimulates protein kinase (PKA), and PKA in turn activates the CFTR protein (Blanchard et al., 2014, FASEB J., vol. 28, p. 791-801). Therefore, it was speculated that increasing cAMP levels, by activation of the adenyl cyclase and/or by inhibition of phosphodiesterase, could restore CFTR-dependent ion transport in cells expressing endogenous ΔF508-CFTR; however, such attempts were generally unsuccessful (Schultz et al., 1999, J. Membr. Biol., vol. 170, p. 51-66; Grubb et al., 1993, Am. J. Respir. Cell Mol. Biol., vol. 8, p. 454-460, reviewed by Blanchard et al., supra).

CFTR Modulators

CFTR-dependent ion transport depends on the amount of (properly folded) CFTR protein at the cell membrane, as well as on the activity of said CFTR protein. Different agents having an effect on the CFTR protein, positive and negative, have been investigated in the past. Based on that research, the pharmaceutical active ingredients that have been tested or proposed to act on the CFTR protein can be categorized into distinct categories: (1) CFTR correctors, i.e. agents that contribute to correcting the levels of the (mutant) CFTR protein at the cell surface, (2) CFTR potentiators, i.e. agents that increase the functionality of the (mutant) CFTR protein at the cell surface, and (3) CFTR amplifiers, i.e. agents that increase the levels of CFTR across all mutation classes (Miller et al., 2016, Am. J. Respir. Crit. Care Med., vol. 193, A 5574), in one theoretical model by stabilization of CFTR mRNA (Molinski et al., 2017, EMBO Molecular Medicine, vol. 9, p. 1224-1243), although the term “CFTR amplifier” as used herein is not limited to said theoretical model. Together, CFTR potentiators, CFTR correctors and CFTR amplifiers are termed “CFTR modulators” (Kuk et al., 2015, Ther. Adv. Resp. Dis., vol. 9, p. 313-326; Molinski et al., 2017, EMBO Molecular Medicine, vol. 9, p. 1224-1243). Combined treatments consisting of a potentiator and/or a corrector and/or an amplifier have also been proposed. Recent progress in the field has shown that the appropriate selection of potentiator and corrector depends inter alia on the genotype of the cystic fibrosis patient to be treated.

In general, CFTR potentiators are agents which influence the activity of the CFTR protein; these molecules require for their functionality that CFTR is as such present at the epithelial cell surface. The pharmaceutical agent ivacaftor (VX 770) is such a potentiator of CFTR channels defective in their chloride/bicarbonate gating or conductance, but present at the epithelial cell surface, such as the CFTR mutant G551D (gating mutant) and R117H (conduction mutant); it increases the open probability of such channels. However, ivacaftor is only approved for pharmaceutical use by its own for treating a few such specific mutations of the CFTR protein, which represent a small subset of the population of patients with cystic fibrosis (Kuk et al., 2015, Ther. Adv. Resp. Dis., vol. 9, p. 313-326).

In general, CFTR correctors are agents that can cause an increase of the number of CFTR molecules on the epithelial cell surface; they are believed to act like chaperones during folding and/or intracellular transport of CFTR. Lumacaftor (VX809) is such a CFTR corrector; however, it has so far not been approved for pharmaceutical use by its own. In general, known CFTR correctors are not cAMP-dependent. Without wishing to be bound to a particular theory, it is presently assumed that some PDE4 inhibitors, such as roflumilast, and the CFTR potentiator ivacaftor (VX-770) elicit a common stimulatory downstream effect on CFTR activation. According to the present understanding in the art, PDE4 inhibitors such as roflumilast are, however, generally not classified as CFTR potentiators.

There have also been attempts to combine the use of different agents in the treatment of cystic fibrosis, or for finding agents that have more than one desired effect in ameliorating the symptoms or fighting the causes of cystic fibrosis; such attempts have so far been hampered by occurrence of side effects or limited to very small patient subgroups. Some examples will be described in the following.

WO 2015/175773 A1 mentions the use of a PDE4 inhibitor in combination with one or more CFTR potentiators, such as ivacaftor, and/or one or more CFTR correctors, such as lumacaftor, but does not provide experimental evidence for any potential advantage associated with such combined use. Specifically for treatment of a subgroup of cystic fibrosis patients, namely those homozygous for ΔF508—although the CFTR potentiator ivacaftor (VX 770) alone was found therapeutically insufficient (Kuk et al., 2015, Ther. Adv. Resp. Dis., vol. 9, p. 313-326)—the combined administration of ivacaftor (VX 770) with the CFTR corrector lumacaftor (VX809), was found satisfactory (Wainwright et al., 2015, N. Engl. J. Med., vol. 373, p. 220-231). No single agent suitable for treating cystic fibrosis patients characterized by at least one mutation in the CFTR gene, which is causative for incorrect processing and/or folding of the CFTR protein, has been identified so far, let alone clinically developed.

Notwithstanding the still incomplete knowledge of cystic fibrosis disease mechanisms, it is widely assumed that cystic fibrosis organ pathology could be alleviated by correction folding defects and/or processing defects of mutant CFTR, thereby restoring functional expression of mutant CFTR (such as ΔF508 CFTR; Lukacs et al., 2012, Trends Mol. Med., vol. 18, p. 81-91).

There is thus still a need for the development of efficient treatments of cystic fibrosis, both at the level of CFTR processing and folding and stability and at the level of CFTR activity (gating/conductance). In particular, there is a need to provide a satisfactory treatment to those subjects which are affected by, or prone to, reduced CFTR processing and/or folding. It has been proposed that an ideal therapy for cystic fibrosis would be a single agent that normalizes mutant CFTR folding, processing, and function to resemble that of wild-type CFTR (Rowe et al., Cold Spring Harb. Perspect. Med., 2013, vol. 3, a009761), however, no such agent has yet been described. For example WO 2015/175773 A1 mentions that CFTR potentiators and/or CFTR correctors could be used in combination with certain further compounds with in vitro PDE4 inhibitory activity, but experimental data for the proposed combined use are not provided and single use is not proposed to be therapeutically effective. Therefore, the search for suitable agents has been ongoing.

### PROBLEM TO BE SOLVED

Thus, an object of the present invention includes eliminating the disadvantages associated with the state of the art. Particular objects comprise the provision of a reliable treatment of cystic fibrosis that is convenient to use and not associated with undue undesired effects, including treatment of subgroups of cystic fibrosis patients for which no fully satisfying therapies are available to date. Various drawbacks of the state of the art define further goals for improvement addressed by the present inventors, and these goals have arrived at by the contribution described and claimed herein.

### SUMMARY OF THE INVENTION

The present invention relates to the treatment of cystic fibrosis. In particular, the present invention is beneficial for the treatment or prevention of cystic fibrosis in subjects characterized by at least one mutation in the CFTR gene which is causative for incorrect folding and/or processing of the CFTR protein. Specifically, the present invention relates to a compound for use in the prevention and/or treatment of cystic fibrosis in a subject, wherein the subject is characterized by at least one mutation in the CFTR gene which is causative for incorrect folding and/or processing of the CFTR protein, and wherein the compound is a compound of general formula (I)

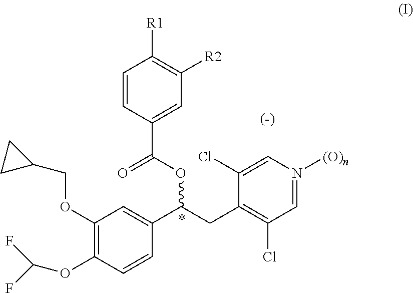

wherein:

n is 0 or 1;

R1 and R2 may be the same or different, and are selected from the group consisting of:
linear or branched C₁-C₆ alkyl, optionally substituted by one or more halogen atoms;
OR3 wherein R3 is a linear or branched C₁-C₆ alkyl optionally substituted with one or more halogen atoms or C₃-C₇ cycloalkyl groups; and
HNSO₂R4 wherein R4 is a linear or branched C₁-C₄ alkyl optionally substituted with one or more halogen atoms,
wherein at least one of R1 and R2 is HNSO₂R4, the pharmaceutically acceptable inorganic or organic salts, hydrates, solvates or addition complexes thereof,
and wherein the compound is the (−) enantiomer.

Preferably, in the compound of general formula (I) for use according to the present invention, R1 is HNSO₂R4; R4 is suitably methyl. Preferably, in the compound of general formula (I) for use according to the present invention, R2 is OR3; R3 is suitably cyclopropylmethyl. Preferably, in the compound of general formula (I) for use according to the present invention, n is 1.

In one embodiment, the compound of formula (I) is a compound wherein R1 is HNSO₂R4, wherein R4 is methyl, R2 is OR3, wherein R3 is cyclopropylmethyl and n is 0.

In one embodiment, the compound of formula (I) is a compound wherein R1 is OR3, R2 is HNSO₂R4, wherein R4 is methyl and n is 1.

In one embodiment, the compound of formula (I) is a compound wherein R1 is methyl, R2 is HNSO₂R4 wherein R4 is methyl and n is 1.

In one embodiment, the compound of formula (I) is a compound wherein both R1 and R2 are HNSO₂R4, wherein R4 is methyl and n is 0.

In one embodiment, the compound of formula (I) is a compound wherein both R1 and R2 are HNSO₂R4, wherein R4 is methyl and n is 1.

The subject in which cystic fibrosis may be prevented or treated according to the present invention is a mammal, preferably a human.

In the present invention the compound of formula (I) is administered to the subject. In particular, all aspects and embodiments of the present invention foresee that the compound of formula (I) is administered to a subject in need thereof. A subject in need thereof is a subject characterized by at least one mutation in the CFTR gene which is causative for incorrect folding and/or processing of the CFTR protein, as described in detail throughout this specification.

In a first specific embodiment, said subject is characterized by at least one mutation in the CFTR gene which is causative for incorrect folding of the CFTR protein. Any mutations of this kind is also referred to herein as “folding mutation”, a term which is applicable both to the protein level and to the level of the nucleic acid that encodes the same. This embodiment includes the mutation ΔF508 on at least one allele. Thus, preferably, in the human subject characterized by at least one mutation of the CFTR gene, the at least one mutation is the mutation ΔF508 encoded by the CFTR gene. More preferably, said human subject, or more precisely the genome of said human subject, is homozygous for the mutation ΔF508.

In a second specific embodiment, said subject is characterized by at least one mutation in the CFTR gene which is causative for incorrect processing of the CFTR protein. Any mutation of this kind is also referred to herein as “processing mutation”, a term which is applicable both to the protein level and to the level of the nucleic acid that encodes the same. The first and the second specific embodiments are not necessarily mutually exclusive.

Preferably, the at least one mutation is a genomic mutation of the CFTR gene. Preferably, the at least one mutation is a mutation of the CFTR gene present in the cells of the respiratory tract of said subject.

In some embodiments, the compound according to general formula (I) for use according to the present invention, also has PDE4 inhibitory activity. Without wishing to be bound to any particular theory, it is however envisaged that the PDE4 inhibition is not necessary and/or not sufficient for the mechanistic explanation of the effect of the compound of general formula (I) on the CFTR protein encoded by a CFTR gene having at least one mutation, according to the present invention.

In some embodiments, said subject suffers from symptoms of cystic fibrosis in the respiratory tract. In some embodiments, said subject suffers from symptoms of cystic fibrosis in the gastrointestinal tract. In some embodiments, said subject suffers from symptoms of cystic fibrosis in the respiratory tract and also in the gastrointestinal tract.

In one embodiment, the compound of general formula (I) is administered by inhalation.

In one embodiment, the compound of general formula (I) is administered by a device selected from a single- or multi-dose dry powder inhaler, a metered dose inhaler and a soft mist nebulizer.

In some embodiments, the compound of general formula (I) is used or administered in combination with at least one second pharmaceutically active component. At least one second pharmaceutically active component is preferably not a compound of general formula (I). In one preferred embodiment, the at least one second pharmaceutically active compound is a CFTR corrector, such as e.g. lumacaftor. In a second preferred embodiment, the second pharmaceutically active compound is a CFTR potentiator, such as e.g. ivacaftor. In a third preferred embodiment, the at least one second pharmaceutically active compound is a combination of a CFTR corrector and a CFTR potentiator; in other words, both a CFTR corrector and a CFTR potentiator can be administered together with the compound of the invention.

### DETAILED DISCLOSURE OF THE INVENTION

The following detailed description discloses specific and/or preferred variants of the individual features of the invention. The present invention also contemplates as particularly preferred embodiments those embodiments, which are generated by combining two or more of the specific and/or preferred variants described for two or more of the features of the present invention.

A person of ordinary skill in the art will appreciate that the invention described herein is susceptible to variations and modifications other than those specifically described. Thus, it will be apparent to the person of ordinary skill in the art that the present disclosure includes all such variations and modifications. The disclosure also includes all of the entities, compounds, features, steps, methods or compositions referred to or indicated in this specification, individually or collectively, and any and all combinations or any two or more of said entities, compounds, features, steps, methods or compositions. Thus, unless specifically stated otherwise herein or the context requires otherwise, reference to a single entity, compound, feature, step, method or composition shall be taken to encompass one and a plurality (i.e. more than one, such as two or more, three or more or all) of those entities, compounds, features, steps, methods or compositions.

The present disclosure is not limited in scope by the specific embodiments described herein, which are provided herein for the purposes of illustration and of exemplification. Functionally or otherwise equivalent entities, compounds, features, steps, methods or compositions are within the scope of the present disclosure.

Unless specifically stated otherwise or the context requires otherwise, each embodiment, aspect and example disclosed herein shall be taken to be applicable to, and combinable with, any other embodiment, aspect or example disclosed herein.

Each of the documents cited herein (including all patents, patent applications, scientific publications, manufacturer's specifications, instructions, presentations, etc.), whether above or below, are hereby incorporated by reference in their entirety. Nothing herein is to be construed as an admission that the present invention is not entitled to antedate a specific teaching.

Unless specifically defined otherwise, all technical and scientific terms used herein have the same meaning as commonly understood by one of ordinary skill in the art (e.g., in genetics, molecular biology, gene expression, cell biology, cell culture, medicine, anatomy, histology, immunology, immunohistochemistry, inorganic and organic chemistry, protein chemistry, and biochemistry). Textbooks and review articles published e.g. in English typically define the meaning as commonly understood by one of ordinary skill in the art.

The expression “and/or”, e.g., “X and/or Y” shall be understood to mean either “X and Y” or “X or Y” and shall be taken to provide explicit disclosure of “and”, of “or” and of both meanings (“and” or “or”).

As used herein, unless specified otherwise, the terms “about”, “ca.” and “substantially” all mean approximately or nearly, and in the context of a numerical value or range set forth herein preferably designates +/−10%, more preferably +/−5%, around the numerical value or range recited or claimed.

Wherever reference is made to an agent, such as to a molecule, then pharmaceutically acceptable inorganic or organic salts, hydrates, solvates or addition complexes thereof are comprised within the scope of the present invention and fully covered by this specification as well as the respective agent itself. Thus, also included in the invention are pharmaceutical compositions that include an agent as described herein and pharmaceutically acceptable carriers or diluents, as well as methods of delivering said agents or compositions to patients by administering to the patients such agents or compositions.

Unless expressly specified otherwise, the word “comprise”, or variations such as “comprises” or “comprising” is used in the context of the present document to indicate that further members may optionally be present in addition to the members of the list introduced by “comprising”. It is, however, contemplated as a specific embodiment of the present invention that the term “comprising” encompasses the possibility of no further members being present, i.e. for the purpose of this embodiment “comprising” is to be understood as having the meaning of “consisting of”.

Unless expressly specified otherwise, all indications of relative amounts regarding the present invention are made on a weight/weight basis. Indications of relative amounts of a component characterized by a generic term are meant to refer to the total amount of all specific variants or members covered by said generic term. If a certain component defined by a generic term is specified to be present in a certain relative amount, and if this component is further characterized to be a specific variant or member covered by the generic term, it is meant that no other variants or members covered by the generic term are additionally present such that the total relative amount of components covered by the generic term exceeds the specified relative amount; more preferably no other variants or members covered by the generic term are present at all.

The term “agent” as used herein, unless specified otherwise, generally refers to a compound or composition, preferably to a compound. An agent is capable of producing an effect on a living organism and/or on a cell from a living organism or derived from a living organism, e.g. by acting on a cell and/or on body tissue, or in an environment. The physical state of an agent is not particularly limited and, unless specified otherwise, may be in the air, water, and/or solid state. The type of agent is not particularly limited, unless specified otherwise, and thus, an agent may be a chemical and/or a biomolecule such as a protein or a nucleic acid. Specific agents defined herein are useful in the present invention.

An “adverse effect”, as used herein, is an undesired harmful effect resulting from an administration of an agent (a drug) to a subject. Adverse effects include, without limitation, morbidity, mortality, alteration in body weight, levels of enzymes, loss of function, or any pathological change detected at the microscopic, macroscopic or physiological level. Adverse effects may cause a reversible or irreversible change, including an increase or decrease in the susceptibility of the individual to other chemicals, foods, or procedures, such as drug interactions.

The term “allele” refers to is a variant form of a given gene (or locus), e.g. in a subject to be treated according to the present invention. The term is applicable to subjects with two sets of chromosomes, i.e. diploid subjects; respective sets of chromosomes are referred to as homologous chromosomes. If both alleles at a gene (or locus) on the homologous chromosomes are the same, the alleles and the organism are “homozygous” with respect to that gene (or locus). If the alleles are different, the alleles and the organism are “heterozygous” with respect to that gene.

An “allelic variant” relates to an alteration in the normal sequence of a gene. Complete gene sequencing often identifies numerous allelic variants for a given gene.

“allelic frequency”, as used herein, refers to the percentage of a particular allele in a given population. For a human allelic frequency, unless specified otherwise, the given population is the total population of humans at the effective date of this specification, irrespective of age, race, ethnic or geographic origin.

The term “cystic fibrosis”, as used herein, has the general meaning used in the art, in its broadest sense; notwithstanding the foregoing, specific aspects of the present invention are directed at a subgroup of subjects affected with “cystic fibrosis”. In general, cystic fibrosis is a condition caused by the presence of mutations in a subject's gene for the cystic fibrosis transmembrane conductance regulator (CFTR) protein, in the present understanding in both the subject's genes (alleles) for the CFTR protein, although the present invention is not necessarily limited to such understanding. “Cystic fibrosis” is normally diagnosed by a sweat test and/or genetic testing (O'Sullivan et al., 2009, Lancet, vol. 373, p. 1891-1904), e.g. by screening of infants at birth and/or by testing of individual subjects e.g. in the case of suspicion by a medical practitioner (O'Sullivan et al., supra). The term “cystic fibrosis”, as used herein, is not limited to a particular type or method of diagnosis.

“CFTR” as used herein, stands for the cystic fibrosis transmembrane conductance regulator, and can stand for the wild type form thereof, as well as any mutant thereof, particularly loss-of-function mutants, unless the context dictates otherwise. “CFTR” is also used herein to refer to the gene encoding a CFTR protein, wild type or mutant.

The term “CFTR modulator”, as used herein is a generic term that refers to an agent that, when contacted with a CFTR-expressing cell or with a subject, can influence the folding and/or processing and/or gating and/or conductance of the CFTR protein. Typically, a CFTR modulator is an agent that targets a defect caused by one or more mutations in the CFTR gene. Examples of CFTR modulators are CFTR correctors, CFTR potentiators and CFTR amplifiers.

The term “CFTR corrector”, as used herein, refers to an agent that, when contacted with a CFTR-expressing cell or with a subject, has an effect to partially or completely overcome defective protein processing that normally results in reduced presence of CFTR and/or of reduced display of CFTR. The term is not limited to any particular mode of action or mechanistic explanation.

The term “CFTR potentiator”, as used herein, refers to an agent that, when contacted with a CFTR-expressing cell or with a subject, has an effect to partially or completely overcome reduced activity of CFTR, such as reduced conductance and/or of reduced gating of CFTR. The term is not limited to any particular mode of action or mechanistic explanation.

The terms “encode”, “encoding” and the like, refer to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence, and the non-coding strand, used as the template for transcription of a gene or cDNA, can be said to be “encoding” the protein or other product of that gene or cDNA.

The terms “express”, “expressed”, and “expression”, “gene expression” and the like, as used herein, relate to the use of information from a gene in the synthesis of a functional gene product. Gene expression comprises at least the transcription, and optionally comprises one of more additional features, optionally selected from the open list comprising RNA editing, translation and post-translational modification. When gene expression is determined, the presence of an expression product, such as non-edited or edited RNA, or even the encoded protein, is determined. The above terms, used in connection with a particular gene or locus, intend to specify the expression of the genetic information from that gene or locus; for example, when it is said that CFTR is expressed, it is meant to say that the CFTR gene is expressed.

As used herein, the term “flow cytometry” refers to a laser- or impedance-based, biophysical technology suitable for cell counting, cell sorting, analysis of cell properties, and biomarker detection (such as, in particular, detection of cell surface molecules, such as Cluster of Differentiation (CD) molecules). Flow cytometry requires cells in suspension; in order to analyze adherent cells, these need to be detached from the substrate, e.g. culture vessel, to which they adhere, e.g. by enzymatic treatment such as trypsinization, by which they become cells in suspension. The cells in suspension, i.e. cells in a stream of fluid, are passed through an electronic detection apparatus (flow cytometry apparatus). The flow cytometry apparatus analyzes the cell, e.g. based upon the specific light scattering of each cell. A commercial flow cytometry apparatus can be used, such as FACSAria III flow cytometer (BD Biosciences). The data generated by flow-cytometers can be plotted in a single dimension, to produce a histogram, or in two-dimensional dot plots or even in three dimensions. Plots may be made using scales of choice, such as linear or logarithmic scales. The regions on these plots can be sequentially separated, based on fluorescence intensity, by creating a series of subset extractions, termed “gates”.

“Fluorescence-activated cell sorting”, or interchangeably “FACS”, as used herein, is a specialized type of flow cytometry. FACS is a method for sorting a heterogeneous mixture of biological cells into two or more populations, based upon the specific light scattering and/or fluorescent characteristics of each cell. The type of fluorophore used as label for FACS is not particularly limited; in some embodiments, fluorophores are attached to an antibody that recognizes a target feature, such as a cell surface protein (such as, in particular, detection of cell surface molecules, such as Cluster of Differentiation (CD) molecules). A fluorophore may alternatively be attached to a chemical entity with affinity for the cell membrane or another cellular structure. Each fluorophore has a characteristic peak excitation and emission wavelength, which is detected by the apparatus suitable for FACS. A commercial apparatus can be used.

The term “heterologous” as used herein describes something consisting of multiple different elements.

The term “loss-of-function” refers to a genetic mutation (i.e. an alteration present in a mutant gene and its product), i.e. a mutation in a gene (or locus) that causes that the product of such gene (typically the protein encoded by such gene) does not function as efficiently as the respective wild type protein, or that the mutation in the gene or locus causes the product of a gene or locus to be expressed at different levels, with a different life time or other different feature that affects the function or production or life time of the product of such gene (or locus). It is important to note that the term “loss-of-function” does not imply or require that function is lost completely, with respect to the wild-type-protein: rather, the term is a relative term which indicates that the function of a loss-of-function mutant is less than 100% (e.g. less than 90%, less than 80%, less than 70%, less than 60%, less than 50%) than the function of the wild-type protein. Normally, the term “loss-of-function” refers to a mutation in the respective haplotype and can be used irrespective of whether or not a second copy that can complement the loss-of-function is encoded by the respective other chromosome of the subject concerned. However, e.g. for recessive loss-of-function mutations, the term may be used to specifically designate that the subject is characterized by two loss-of-function copies of the respective gene (or locus) and therefore lacks the normal functionality of the gene product. A loss-of-function mutation of the CFTR gene may be a mutation that affects gating and/or conductance (gating/conductance mutation) and/or a mutation that affects folding and/or processing (folding/processing mutation). An exemplary loss-of-function mutation of the CFTR gene is a mutation causing the ΔF508 mutation at protein level. Without wishing to be bound to any particular theory, the ΔF508 mutation of the CFTR protein is normally considered to be a recessive loss-of-function mutation.

The terms “multi” and “multiple” as used herein mean a multitude, i.e. any number of two or more.

The term “mutation”, as used herein, refers to the alteration of the nucleotide sequence of the genome of an organism, virus, or extrachromosomal DNA or other genetic elements. The term also extends to mutations of an amino acid sequence, particularly the amino acid sequence of a gene that carries at least one (non-silent) mutation. Unless specified otherwise, a mutation of the nucleotide sequence is a permanent alteration. Mutations present in the germ line are normally inheritable. In general, a mutation of the nucleotide sequence can result in many different types of change in sequences: mutations in genes can either have no effect, alter the product of a gene, or prevent the gene from functioning properly or completely. Mutations can also be present in non-genic regions. Unless specified otherwise, the wild type sequence is used as a reference sequence to describe a mutation. Thus, for example, when it is said that a given mutant is characterized by mutation of position 508 of a polypeptide sequence, this indicates that at position 508 the mutant does not have the same amino acid as the wild type polypeptide. Specific types of mutations of a nucleotide sequence and/or an amino acid sequence include alterations such as deletions, substitutions, additions, insertions and splice variants. A “deletion” with respect to a nucleotide sequence refers to the absence of one or more nucleotide(s) in the nucleotide sequence. A “deletion” with respect to an amino acid sequence refers to the absence of one or more amino acid residue(s) in the polypeptide. An “addition” with respect to a nucleotide sequence refers to the presence of one or more additional nucleotide(s) in nucleotide sequence. An “addition” with respect to an amino acid sequence refers to the presence of one or more additional amino acid residue(s) in the related polypeptide. A “substitution” with respect to a nucleotide sequence refers to the replacement of one or more nucleotide(s) by (an) other nucleotide(s) in the nucleotide sequence. A “substitution” with respect to an amino acid sequence refers to the replacement of one or more amino acid residue(s) by (an) other amino acid residue(s) in the polypeptide. Additions, deletions and substitutions to a nucleotide sequence, such as to an open reading frame, may be 5′ terminus, the 3′ terminus, and/or internal. Additions, deletions and substitutions to a polypeptide, may be at the amino terminus, the carboxy terminus, and/or internal. An “insertion” with respect to a nucleotide sequence and/or a polypeptide sequence is an addition of one or more nucleotides, or one or more amino acid residues, respectively, specifically at an internal position of the respective sequence. The term “splice variant” is used to describe that the RNA encoding a polypeptide sequence is spliced differently from the respective wild type RNA, typically as a result of a mutation at nucleic acid level, usually resulting in a polypeptide translation product which is different from the wild type polypeptide. The term “splice variant” can be used not only with respect to the respective RNA, but also with respect to the respective template DNA sequence (typically genomic DNA) and with respect to the sequence of the polypeptide encoded by such RNA.

The term “mutant” is generally intended to refer to a nucleic acid sequence or amino acid sequence which is different from the wild type sequence. In cases where polymorphisms at the nucleic acid sequence exist which are, however, not reflected at the level of the respective encoded polypeptide (silent mutations, degeneracy of the genetic code), the term “mutant”, on nucleic acid level, specifically refers only to those nucleic acid variants which encode a mutant polypeptide. Mutants can contain different combinations of mutations, alone or in combination, including more than one mutation and different types of mutations.

The term “peptide” according to the invention comprises oligo- and polypeptides and refers to substances comprising two or more, preferably 3 or more, preferably 4 or more, preferably 6 or more, preferably 8 or more, preferably 10 or more, preferably 13 or more, preferably 16 more, preferably 21 or more and up to preferably 8, 10, 20, 30, 40 or 50, in particular 100 amino acids joined covalently to a chain by peptide bonds.

The term “protein” preferably refers to large peptides, preferably to peptides with more than 100 amino acid residues, but in general the terms “peptide”, “polypeptide” and “protein” are synonyms and are used interchangeably herein, unless the context dictates otherwise.

The term “pharmaceutically acceptable” generally describes that a certain substance can be administered to a subject, optionally and preferably in combination with an agent, without the agent causing intolerable adverse effects, at the dosage used.

The term “pharmaceutically acceptable carrier” is used to refer to any one or more of solvents, dispersion media, coatings, antibacterial and antifungal agents, isotonic and absorption delaying agents, and the like that are physiologically compatible and are suitable for administration to a subject for the methods described herein. Examples of such pharmaceutically acceptable carriers comprise without limitation one or more of water, saline, phosphate buffered saline, dextrose, glycerol, ethanol and the like, as well as combinations thereof. Particularly for the case of liquid pharmaceutical compositions, it may be preferable to include isotonic agents, for example, sugars, polyalcohols such as mannitol, sorbitol, or sodium chloride in the composition. Pharmaceutically acceptable carriers may further comprise auxiliary substances such as wetting or emulsifying agents, preservatives or buffers, which enhance the shelf life or effectiveness of the agent. A pharmaceutically acceptable carrier is typically comprised in a composition according to the present invention.

The term “pharmaceutically active agent” refers to an agent that can be used in the treatment of a subject where the agent would be of benefit, e.g., in ameliorating the symptoms of a disease or disorder. In addition, a “pharmaceutically active agent” can have a positive or advantageous effect on the condition or disease state of a subject when administered to the subject in a therapeutically effective amount. Preferably, a pharmaceutically active agent has curative properties and may be administered to ameliorate, relieve, alleviate, reverse, delay onset of or lessen the severity of one or more symptoms of a disease or disorder. A pharmaceutically active agent may have prophylactic properties and may be used to delay the onset of a disease or to lessen the severity of such disease or pathological condition. For example, an agent of the invention is considered herein as a pharmaceutically active ingredient for the treatment of cystic fibrosis, as claimed. In another example, a pharmaceutically active protein can be used to treat a cell or an individual which does not normally express a protein, or not at the desired levels, or which mis-expresses a protein, e.g., a pharmaceutically active protein can compensate for a mutation, or for lack of sufficiently high expression, by supplying a desirable protein. The term “pharmaceutically active peptide or protein” includes entire proteins or polypeptides, and can also refer to pharmaceutically active fragments thereof. It can also include pharmaceutically active analogs of a peptide or protein.

An “open reading frame” or “ORF” is a continuous stretch of codons beginning with a start codon and ending with a stop codon.

When it is said herein that a protein is “present”, e.g. in a cell, this is meant to specify that a protein exists in the cell at levels which are determinable by methods according to the state of the art. Such a protein, e.g. the CFTR protein, is typically the expression product of a gene of that cell. Thus, determination of the presence of a protein is an indirect way of determining the expression of the respective gene.

When it is said herein that a protein is “displayed”, e.g. on a cell, this is meant to specify that a protein exists at the surface of a cell at levels which are determinable by methods according to the state of the art. Thus, determination of the display of a specific protein on the cell surface is a specific way of determining the presence of said protein.

According to the present invention, RNA may encode a peptide or protein. Accordingly, RNA may contain a coding region (open reading frame (ORF)) encoding a peptide or protein. For example, RNA may encode and express an antigen or a pharmaceutically active peptide or protein. Unless specified otherwise, the term RNA may be used herein both for primary RNA transcripts as well as for spliced RNA, including any splicing variants, as described herein.

According to the present invention, the term “respiratory tract” generally refers to the part of the anatomy of the respiratory system involved with the process of respiration. Thus, the respiratory tract includes without limitation nose mouth, nasal cavity, pharynx, larynx, epiglottis, trachea, lungs, primary (main) bronchi, secondary (lobar) bronchi, tertiary (segmental) bronchi, small airways (also called bronchioles), and alveoli (thin specialized structures that function in gas exchange).

The term “gastrointestinal tract”, as used herein, generally refers to the collection of anatomic structures or series of connected body organs which takes in food, digests it to extract and absorb energy and nutrients, and expels the remaining waste as feces. The gastrointestinal tract of a mammal comprises without limitation the mouth, oesophagus, stomach, and intestines.

The term “subgroup” (symbol H), as used herein, refers to a proper subgroup of a group G. I.e. a subgroup H is a proper subset of G (i.e. H≠ G). This is usually represented notationally by H<G, read as “H is a proper subgroup of G”. If H is a subgroup of G, then G is called an overgroup of H. A “patient subgroup” is a subgroup of patients suffering from a condition. For example, a subgroup of cystic fibrosis patients is a subset of all cystic fibrosis patients.

The terms “subject” and “patient”, as used herein, relate to a mammal. For example, mammals in the context of the present invention are humans, non-human primates, domesticated animals including but not limited to dogs, cats, sheep, cattle, goats, pigs, horses etc., laboratory animals including but not limited to mice, rats, rabbits, etc., as well as animals in captivity such as animals of zoos. The terms “subject” and “patient” as used herein particularly include humans. The subject (human or animal) has two sets of chromosomes; that is, the subject is diploid. The term “patient” refers to a subject which suffers from a condition, is at risk of suffering from a condition, has suffered from a condition, or is predicted to suffer from a condition, and which may be subjected to therapy, e.g. by administration of an agent. The patient's condition may be chronic and/or acute. Thus, a “patient” can also be described as a subject subjected to a therapy and/or or in need of a therapy.

The term “therapy” is to be understood broadly and refers to the treatment of a subject with the goal to prevent or treat a condition in the subject. In preferred embodiments, therapy specifically includes the administration of an agent to the subject.

In the context of the present invention, the term “transcription” refers to a process wherein the genetic code in a DNA sequence is transcribed into RNA.

The term “translation” according to the invention refers to the process by which a messenger RNA directs the assembly of a sequence of amino acids on the ribosomes of a cell to make a peptide or protein.

The term “wild type” is used herein to refer to an allele, e.g. of the CFTR gene, that is not associated with cystic fibrosis, i.e. an allele that is understood to contribute to the typical phenotypic character as seen in “wild” populations of subjects. An allele that is not “wild type” is referred to herein as “mutant” or “mutated”, or the like.

The present invention is based on several findings, which are interrelated and thus together lead the inventors to arrive at the various aspects of the invention, which will all be described individually in the following. All aspects of the present invention are based inter alia on the finding that the compound of formula (I) is beneficial for the treatment and prevention of cystic fibrosis in a specific patient subgroup.

New Treatment For a Specific Patient Subgroup

The present invention offers a new prevention or treatment for a specific subgroup of cystic fibrosis patients. According to the present invention, the use of a compound according to general formula (I) of the present disclosure for the treatment of cystic fibrosis in subjects associated with one or more loss-of-function mutations of the CFTR gene is provided. The new use of such compound is based on specific findings reported herein.

The present invention also relates to a method of treating a patient suffering from cystic fibrosis, wherein the patient is characterized by at least one mutation in the CFTR gene which is causative for incorrect folding and/or processing of the CFTR, wherein the method comprises administering an effective amount of a compound of general formula (I) to the patient. The terms “patient” and “subject” are used interchangeably herein, particularly with reference to a patient/subject characterized by at least one mutation in the CFTR gene which is causative for incorrect folding and/or processing of the CFTR.

FIG. 8 and SEQ ID NO: 1 provide the amino acid sequence of the wild-type human CFTR protein (1480 amino acids). Accession P13569, version P13569.3, dbsource UniProtKB: locus CFTR_HUMAN. Without wishing to be bound to any particular theory, it is understood that the subjects that will particularly profit from the prevention or treatment according to the present invention are those subjects which are characterized by at least one mutation in at least one allele of the human CFTR protein.

As an introductory comment, in view of the paucity of structural information on full-length wild type and mutant CFTR, as well as the complexity of the defects caused by some genetic mutations of the CFTR gene (e.g. the mutation causative for ΔF508), the drug discovery in cystic fibrosis largely relies on phenotypic assays based on CFTR channel function. Some specific halide-sensing fluorescent protein mutants, namely yellow fluorescent protein (YFP) mutants whose fluorescence is strongly quenched (reduced) by iodide (Jayaraman et al., 2000, J. Biol. Chem., vol. 275, p. 6047-6050) is valuable, because iodide is a halide that is efficiently transported by CFTR (Rowe et al., Cold Spring Harb. Perspect. Med., 2013 vol. 3, a009761). In addition to that, immunophenotypic approaches that detect the total presence of (mutant) CFTR protein (e.g. Western Blot) and/or the display of CFTR protein at the cell surface (e.g. immunostaining, optionally combined with FACS and/or microscopy) are helpful.

In contrast to traditional cystic fibrosis therapies, such as antibiotics, mucolytics, anti-inflammatory agents and e.g. nebulized hypertonic saline, which treat CF disease manifestations, the compound of the present invention directly addresses the underlying CFTR anion channel defect. The data reported in Example 2, as discussed herein, make plausible that the compound according to general formula (I) has CFTR corrector function. These findings are completely surprising: while recent literature suggests that the PDE4 inhibitor roflumilast acts as potentiator of the CFTR protein, i.e. by enhancing the activity of mutant CFTR protein, characterized by specific mutations found in cystic fibrosis patients activity in the airway epithelium (Blanchard et al., 2014, FASEB J., vol. 28, p. 791-801; Lambert et al. Am. J. Respir. Cell Mol. Biol., 2014 vol. 50, p. 549-58), known PDE4 inhibitors have not been described to have the capacity to correct the presence of CFTR protein in cells of cystic fibrosis patients or in in vitro models thereof, let alone to have a causative action (for comparison with the present invention see also e.g. WO 2015/175773 A1). In the art, such as e.g. in WO 2015/175773 A1 and in Blanchard et al., 2014, supra, no evidence of the action of proposed PDE4 inhibitors on the levels of CFTR protein, let alone correction thereof as a causative effect, is shown, let alone proposed. In view of the art, the present inventors' finding, i.e. that the compounds of the present invention have the capacity to act as CFTR correctors in subjects associated with specific mutations of CFTR was unexpected.

In addition to that, Example 1 of the present specification suggests a potentiator function of the compound of general formula (I). Further, it is confirmed in Example 1 that the known PDE4 inhibitor roflumilast has an effect as CFTR potentiator. According to the literature this effect of PDE4 inhibitors, such as roflumuilast, on cystic fibrosis, is strictly related to their capacity to lead, through inhibition of phosphodiesterase 4, to an increase in the concentration of cAMP, in specific cell compartments. An effect of roflumilast (a reference PDE4 inhibitor) is confirmed in Example 1 herein.

As confirmed in Example 1, not only roflumilast but also a compound according to general formula (I) partially restored the activity of mutated CFTR in airway epithelium similar to the potentiator ivacaftor (reference) and the known PDE4 inhibitor roflumilast, which provides evidence that the compound works as a potentiator.

Thus, according to the present invention, a compound of general formula (I) is provided for therapy of a human or animal suffering from cystic fibrosis or prone to suffer from cystic fibrosis. Thus, cystic fibrosis may be prevented or treated in that human or animal based on the present invention.

The examples herein report that a compound of general formula (I) can restore CFTR-dependent ion transport in cells expressing endogenous ΔF508-CFTR; and thus provide evidence that a compound of general formula (I) has a distinguished and beneficial effect on ΔF508-CFTR, other than PDE4 inhibitors previously tested in the art (Schultz et al., 1999, J. Membr. Biol., vol. 170, p. 51-66; Grubb et al., 1993, Am. J. Respir. Cell Mol. Biol., vol. 8, p. 454-460, reviewed by Blanchard et al., supra).

CFTR Correction and Experimental Detection Thereof

Preferably, the compound according to the present invention has CFTR corrector activity. In particular, it is preferred that the compound has CFTR corrector activity in a cell or in a subject characterized by at least one mutation in the CFTR gene which is causative for incorrect folding and/or processing of the CFTR protein. In a particular embodiment, the compound according to the present invention is causative for increasing the presence and/or surface display of the CFTR protein in a cell of such subject.

In the context of the present invention, presence and surface display of the CFTR protein are important. The CFTR protein is a member of the ATP-binding cassette (ABC) transporter superfamily of membrane proteins. The wild type CFTR protein has 1480 amino acid residues (168.142 kDa). The amino acid sequence of the wild type CFTR protein is represented by UniProtKB locus CFTR_HUMAN and is shown in FIG. 14. When correctly inserted into the cell membrane, the CFTR protein functions as a chloride channel and controls the regulation of other transport pathways. Mutations in this gene are associated inter alia with the autosomal recessive disorder cystic fibrosis. Alternatively spliced transcript variants have been described, many of which result from mutations in the CFTR gene.

In the present invention, the presence of the CFTR protein in a cell, particularly on the cell surface, can be corrected. This is due to the newly identified and unexpected function of the compound of general formula (I). Indeed, it is preferred and also demonstrated by the experimental examples herein that achieving CFTR correction is an integral part of the invention as claimed herein. Indeed, attaining the claimed therapeutic effect is a functional technical feature of the present invention. The examples herein make plausible that said functional technical feature is achievable as a direct result of administration of a compound of general formula (I). In other words, the present inventors have identified that a compound of general formula (I) is causative for achieving CFTR correction in a cell or in a subject characterized by at least one mutation in the CFTR gene which is causative for incorrect folding and/or processing of the CFTR protein.

For that purpose, in the context of the present invention, the presence of a protein, such as the CFTR protein, can be determined. More preferably, the presence of a protein on the cell surface, i.e. surface display, is determined. In other words, it is determined whether a protein, such as the CFTR protein, is displayed on the cell surface.

Cells displaying a particular protein on the cell surface can be analyzed e.g. by immunologically active molecules, such as specific antibodies and other immunoreactive molecules. “Cell surface” is used herein in accordance with its normal meaning in the art, and thus specifically includes the outside of the cell which is accessible to binding by proteins and other molecules. A protein is displayed on the surface of cell if it is at least partially located at the surface of said cell and is accessible to binding by antigen-binding molecules such as antigen-specific antibodies added to the cell. In one embodiment, a protein displayed on the surface of cell is an integral membrane protein having an extracellular portion that can be recognized by an antibody. The term “extracellular portion” or “exodomain” in the context of the present invention means a part of a molecule, particularly a protein, that faces the extracellular space of a cell and preferably is accessible from the outside of said cell, e.g., by binding molecules such as antibodies located outside the cell. Preferably, the term refers to one or more extracellular loops or domains or a fragment thereof. The term “portion” is used herein and refer to a continuous or discontinuous element of a structure such as an amino acid sequence. A portion or part of a protein sequence preferably comprises at least 5, in particular at least 8, at least 12, at least 15, at least 20, at least 30, at least 50, or at least 100 consecutive and/or non-consecutive amino acids of the amino acid sequence making up the protein.

A protein detectable by an antibody or other immunoreactive molecule may also be referred to as an antigen. In some embodiments, the cell of the invention may be characterized by displaying—or not displaying—one or more specific antigens. In the context of the present invention, an antigen of the CFTR protein is preferably displayed on the surface of the cell.

In line with the general principles of cell biology, when an antigen is specifically detectable by an antibody or other immunoreactive molecule, e.g. on the surface of an (intact) cell (e.g. by immunostaining) or in lysate of the cell (e.g. by Western Blot), then the gene encoding the antigen (polypeptide) is expressed by the cell. Therefore, detection of an antigen (polypeptide) that is displayed on the surface of the cell is an indirect means for showing that the gene encoding the polypeptide is expressed. Another indirect way for showing that the gene encoding the protein is expressed, and thus present in the cell, is by Western Blot (see e.g. Example 2).

According to the invention, an antigen is displayed on a cell if the level of expression is above the detection limit and/or if the level of expression is high enough to allow binding by antigen-specific antibodies added to the cell. According to the invention, an antigen is said to be not expressed on a cell if the level of expression is below the detection limit and/or if the level of expression is too low to allow binding by antigen-specific antibodies added to the cell. Preferably, an antigen expressed in a cell is expressed or exposed, i.e. is present, on the surface of said cell and, thus, available for binding by antigen-specific molecules such as antibodies or other immune reactive molecule added to the cell. In some cases, a secondary molecule that aids in the detection, such as e.g. an optionally labelled secondary antibody, is also added.

An antibody or other immune reactive molecule may recognize an epitope on the cell. The term “epitope” refers to an antigenic determinant in a molecule such as an antigen, i.e., to a part in or fragment of the molecule that is recognized, i.e. bound, by the immune system, for example, that is recognized by an antibody or other immunoreactive molecule. Detection of an epitope specific for any particular antigen normally allows to conclude that that particular antigen is present on the cell being analyzed.

In one embodiment, a cell, or a sample from the subject, can be characterized by immunophenotyping. “Immunophenotyping” generally means that the cell or sample can be characterized by antigen-specific molecules such as antibodies or other immune reactive molecules, which are added to the cell to determine if an antigen is present. Immunophenotyping includes cell sorting using various methods including flow cytometry, as well as analytic methods on lysed cells and lysed samples, such as Western Blotting. One method for immunophenotyping is flow cytometry, in particular FACS: an analyte, in particular a cell surface protein, is recognized, normally with an antibody or other immunoreactive molecule. The antibody or other immunoreactive molecule is either fluorophore-labelled itself, or recognized by a fluorophore-labelled secondary antibody or other immunoreactive molecule, which is added for that purpose.

Characterization of the Patient Subgroup

The present invention is particularly suitable for a subgroup of subjects suffering from cystic fibrosis, wherein said subgroup is characterized by a specific genotype and a specific phenotype. Regarding the specificity of the genotype, the subject is characterized by at least one mutation in at least one allele of the CFTR gene. Regarding specificity of the phenotype, the mutation is causative for incorrect folding and/or processing of the CFTR protein. Thus, the genetic mutation is a loss-of-function mutation. Nearly 2000 mutations in the CFTR gene have been identified that produce the loss-of-function phenotype by impairing transcription and/or translation, cellular folding and/or processing, and/or chloride channel gating. In general, loss-of-function mutations of the CFTR gene have been described inter alia by Rowe et al. (Cold Spring Harb. Perspect. Med., 2013, vol. 3, a009761) and http://www.genet.sickkids.on.ca/app.

In particular, the present invention relates to a compound of general formula (I) for use in the prevention and/or treatment of cystic fibrosis in a subject, wherein the subject is characterized by at least one mutation in the CFTR gene which is causative for incorrect folding and/or processing of the CFTR protein.

Thus, said at least one mutation in the CFTR gene is not a silent mutation: it is a loss-of-function mutation which is causative for a mutation of the amino acid sequence of the encoded CFTR protein.

Although it has been observed that certain mutations in the CFTR gene are more frequent subjects of Northern European origin or ancestry and less common in subjects with African and Asian origin or ancestry (O'Sullivan, et al., 2009, Lancet, vol. 373, p. 1891-1904), the present invention is applicable irrespective of age, race, ethnic or geographic origin of a subject, unless the context clearly dictates otherwise.

The present invention is in part based on the surprising finding that unexpectedly a compound of general formula (I) as defined herein, such as CHF6001, increases the presence of mutated CFTR protein at the level of the cellular plasma membrane. This is shown in Example 2.

In one embodiment, the subject is characterized by two alleles of mutated CFTR, as described herein. The two mutated alleles may be identical or different. In a preferred embodiment, the two mutated alleles share at least one mutation which is causative for incorrect folding and/or processing of the CFTR protein.

The subject in which cystic fibrosis may be prevented or treated according to the present invention is preferably a mammal, more preferably a human. Examples of non-human animals are slaughter animals and other farm-bred animals such as cattle, pigs, sheep or poultry.

In non-human animal subjects, the present invention is applicable to subjects of subgroups having species homologs of the human CFTR proteins described herein. In general, a “species homolog” is a nucleic acid or amino acid sequence or mutation thereof with a different species of origin from that of a given nucleic acid or amino acid sequence or mutation thereof. Thus, a species homo log of the human CFTR protein is a CFTR protein from a non-human species, and a species homolog of the human mutation ΔF508 in a non-human animal refers to the deletion of a section of the CFTR protein in the non-human animal that corresponds, by sequence homology, the human mutation ΔF508.

The fact that a compound of the present invention is specifically capable of increasing the presence of mutated CFTR protein (Example 2), suggests, without wishing to be bound by any particular theory, that a molecular mechanism other than, or at least in addition to, PDE4 inhibition is responsible for the correction of the cellular processing defect of the CFTR channel observed upon exposure to CHF6001 in Example 2, i.e. that the corrector activity of CHF6001 on the mutated CFTR protein may be due to a different mechanism of action. Such mechanism of action has not yet been fully elucidated at molecular level, but is suggested by the scientific finding reported herein.

Therefore, the present invention is beneficial for the treatment or prevention of cystic fibrosis in subjects characterized by at least one mutation in the CFTR gene which is causative for incorrect folding and/or processing of the CFTR protein. In some embodiments, such subjects may suffer from cystic fibrosis in the respiratory tract and or in the gastrointestinal tract. Subjects that benefit from treatment or prevention according to the present invention represent a subgroup of cystic fibrosis patients. This subgroup, which is defined both genotypically and phenotypically, is narrow and specific compared to the not specifically defined total of cystic fibrosis patients, as mentioned e.g. in WO 2010/089107 A1. In one embodiment, the compound according to the present invention is useful particularly for treating subjects suffering from cystic fibrosis in the respiratory tract. Example 2 evidences that the compound of the present invention is particularly suitable for treating cells of the respiratory tract. Indeed, according to the common general knowledge it is widely accepted that drug discovery in the field of cystic fibrosis of the respiratory tract can largely rely on phenotypic assays based on CFTR channel function (Rowe et al., Cold Spring Harb. Perspect. Med., 2013 vol. 3, a009761). In some embodiments, the subject is associated with a condition selected from pulmonary inflammation. Without wishing to be bound to a particular theory, it is normally understood that the mutation ΔF508 del is a folding mutation.

The subject is characterized by at least one mutation which is characterized by a permanent alteration of the subject's nucleotide sequence, preferably of the subject's genomic nucleotide sequence. Thus, preferably, the at least one mutation is a genomic mutation of the CFTR gene. Preferably, such mutation is a loss-of-function mutation. More preferably, the subject to be treated is characterized by a loss-of-function mutation of the CFTR gene on each of the alleles of the CFTR gene. In other words, at least one allele of the CFTR gene of the subject does not encode a wild type CFTR protein, preferably both alleles of the CFTR gene of the subject do not encode a wild type CFTR protein. Preferably at least one allele of the CFTR gene of the subject encodes a CFTR protein characterized by altered folding, processing, conductance or gating, compared to a wild type CFTR protein. As used herein, a CFTR protein characterized by altered folding, processing, conductance or gating, compared to a wild type CFTR protein may be characterized by a loss-of-function mutation. More preferably both alleles of the CFTR gene of the subject encode a CFTR protein characterized by altered folding, processing, conductance or gating, compared to a wild type CFTR protein. The two alleles of the subject which encode a non-wild type CFTR protein, preferably a CFTR protein characterized by altered folding, processing, conductance or gating, compared to a wild type CFTR protein, may be the same or different. In one preferred embodiment, the two alleles of the subject which encode a non-wild type CFTR protein encode the same mutant of the CFTR protein and optionally have the same nucleotide sequence. Thus, in some preferred embodiment, the subject is homozygous for a mutation in the CFTR gene. Example 1 and Example 2 show that a compound according to general formula (I) is suitable in cells homozygous for a mutation in the CFTR gene.

The at least one mutation in the CFTR gene is selected from a missense mutation (including a non-in-frame insertion or deletion), a frameshift mutation, a splicing mutation, a nonsense mutation, an in frame insertion or deletion (in/del) of one or more amino acids, a promoter mutation, a mutation that affects glycosylation of the CFTR protein, or any other mutation of the CFTR gene that affects the CFTR protein. Preferably, the mutation is an in frame insertion or deletion (in/del) of one or more amino acids. An example thereof is the deletion of the amino acid residue phenylalanine 508 (Phe508, F508), caused by a 3 nucleotide deletion (i.e. in frame). This specific deletion (ΔF508) causes a protein folding defect. If this defect is overcome as provided in the present invention, then the protein can form a functional CFTR channel.

The at least one mutation of the CFTR gene may be a mutation within exon 1 or exon 2 or exon 3 or exon 4 or exon 5 or exon 6 or exon 7 or exon 8 or exon 9 or exon 10 or exon 11 or exon 12 or exon 13 or exon 14 or exon 15 or exon 16 or exon 17 or exon 18 or exon 19 or exon 20 or exon 21 or exon 22 or exon 23 or exon 24 or exon 25 or exon 26 or exon 27 of the CFTR gene. Alternatively or additionally, the at least one mutation of the CFTR gene may be a mutation within intron 1 or intron 2 or intron 3 or intron 4 or intron 5 or intron 6 or intron 7 or intron 8 or intron 9 or intron 10 or intron 11 or intron 12 or intron 13 or intron 14 or intron 15 or intron 16 or intron 17 or intron 18 or intron 19 or intron 20 or intron 21 or intron 22 or intron 23 or intron 24 or intron 25 or intron 26 of the CFTR gene, and/or a mutation that overlaps multiple exons and/or introns. In preferred embodiments, at least one mutation is found in exon 11 of the CFTR gene, i.e. the exon encoding phenylalanine 508 in wild type CFTR (http://www.genet.sickkids.on.ca/CftrDomainPage.html?domainName=NBD1).

Preferably, the at least one mutation of the CFTR gene is a nucleotide mutation which causes a mutation on amino acid sequence level within a nucleotide binding domain (NBD) of the CFTR protein. The NBDs contain a number of highly conserved motifs predicted to bind and hydrolyze ATP. Site directed mutagenesis at these motifs have indicated that ATP binds to both NBDs to control the gating of the channel. In preferred embodiments, at least one mutation is causative for a mutation on amino acid level in the first (more N-terminal) nucleotide binding domain (NBD) of the CFTR protein. Phenylalanine 508 in wild type CFTR is found in the first nucleotide binding domain (NBD1; see http://www.genet.sickkids.on.ca/CftrDomainPage.html?domainName=NBD1).

In one embodiment, the subject to be treated according to the present invention is characterized by at least one mutation in the CFTR protein which is not only a gating mutation or a conductance mutation. For the avoidance of doubt, although phenylalanine 508, in wild type CFTR protein, is located in NBD1, the deletion of phenylalanine 508 does not only cause a defect on gating and conductance, but also on folding of the CFTR protein, as described below.

In preferred embodiments, the subject is characterized by absence of phenylalanine 508, with reference to the wild-type CFTR sequence. Phenylalanine 508 may be absent due to a variety of different alternative genetic mutations, and all such all alternatives are comprised by the present invention, unless the context clearly dictates otherwise. In particular, the at least one mutation in the CFTR gene causing absence of phenylalanine 508 is selected from a missense mutation (including a non-in-frame insertion or deletion), typically at a position in the nucleotide sequence which codes for phenylalanine 508 or upstream of that position; a frameshift mutation, typically at a position in the nucleotide sequence which codes for phenylalanine 508 or upstream of that position; a splicing mutation typically affecting at least any one of exons 1 to 11 and/or introns 1 to 10, a nonsense mutation, typically at a position in the nucleotide sequence which codes for phenylalanine 508 or upstream of that position; an in frame insertion or deletion (in/del) of one or more amino acids, typically at a position in the nucleotide sequence which codes for phenylalanine 508 or upstream of that position. “upstream” in the context of the present invention, has the typical meaning in the field of molecular biology and, when used with reference to a nucleic acid sequence, is intended to specify a position closer to the 5′ end of that nucleic acid sequence.

In a first specific embodiment, said subject is characterized by at least one mutation in the CFTR gene which is causative for incorrect folding of the CFTR protein. Any mutation of this kind is also referred to herein as “folding mutation”, a term which is applicable both to the protein level and to the level of the nucleic acid that encodes the same.

Unless corrected, e.g. by administration of a suitable CFTR corrector, a folding mutation is usually causative for a reduced presence of the CFTR protein in the cell, particularly reduced display of the CFTR protein at the cell surface. Presence of the protein may be detectable, for example, by gel electrophoresis and Western Blot. Display of the protein at the cell surface may be detectable e.g. by immunostaining.

Preferably, when at least one allele (first allele) of the subject to be treated according to the present invention is characterized by a folding mutation, then the second allele is not an allele which is capable to trans-complement the folding defect caused by the folding mutation on the first allele (Cormet-Boyaka et al., 2004, Proc. Natl. Acad. Sci. USA, vol. 101, p. 8221-8226). Preferably, in this embodiment, the subject to be treated according to the present invention encodes a CFTR protein with a folding mutation (same or different) on each of the two alleles of the CFTR gene. When the folding mutation is identical on both alleles, which is preferred, then the subject is homozygous for said folding mutation.

In the context of the present invention, a “folding mutation” refers to a mutation of the CFTR polypeptide sequence (and thus, also to a nucleic acid sequence encoding the same), wherein the mutation of the CFTR polypeptide sequence is causative for inefficient folding of the CFTR polypeptide. Without wishing to be bound to any particular theory, it is presently understood that the folding of CFTR occurs at the endoplasmic reticulum, co- and/or post-translationally; a folding mutation is a mutation is a mutation wherein the mutant CFTR protein does not fold as efficiently as wild type CFTR protein, e.g. due to one or more of the following defects: inefficient formation of the native conformation at the endoplasmic reticulum (ER), inefficient exit from the ER, and/or inefficient glycosylation in the Golgi compartment (Lukacs et al., 2012, Trends Mol. Med., vol. 18, p. 81-91). “does not fold as efficiently as wild type CFTR protein” means that less than 100% of individual CFTR polypeptides are folded as efficiently as wild type CFTR protein, even if a certain percentage of individual CFTR polypeptides should actually be folded correctly. Without wishing to be bound to any particular theory, it is envisaged that partially folded channels are disposed of by ER-associated degradation (ERAD) via the ubiquitin-proteasome system (UPS; Lukacs et al., supra), which explains why the presence and/or surface display of a CFTR folding mutant is usually less than 100%, compared to the levels of presence and/or surface display of wild type CFTR protein, in the respective cell type. Although the underlying cause of the inefficient folding of mutant CFTR has not been terminally clarified and is not critical to the practice of the present invention, it has been proposed that the energetic instability of individual domains of the CFTR protein, the slow domain assembly, and the relatively fast ERAD kinetics all contribute to inefficient folding (Lukacs et al., supra).

The present invention is applicable to any folding mutant of CFTR, unless the context clearly dictates otherwise. In particular, the present invention is applicable to the deletion of phenylalanine 508, with respect to wild-type human CFTR. This mutation can be referred to as ΔF508, ΔPhe508, F508del or Phe508del, or the like. The mutation ΔF508 is the most well studied folding mutation of the human CFTR protein. In a human subject foreseen to be treated according to the present invention, the mutation ΔF508 is present on at least one allele. Thus, preferably, in the human subject characterized by at least one mutation of the CFTR gene, the at least one mutation is the ΔPhe508 in the CFTR gene. Preferably, when at least one allele (first allele) of the subject to be treated according to the present invention is characterized by the folding mutation ΔPhe508, then the second allele is not an allele which is capable to trans-complement the folding defect caused by the folding mutation ΔPhe508. In line with the above, the present invention thus provides the use of a compound according to general formula (I) in a human subject, wherein the genome of said human subject encodes at least the mutation ΔF508 in the CFTR protein.

More preferably, said human subject is homozygous for the mutation ΔPhe508. In that regard, the present invention provides the use of a compound according to general formula (I) in a human subject, wherein the genome of said human subject encodes the mutation ΔF508 in both genomic alleles of the gene encoding the CFTR protein. In other words the present invention provides the use of a compound according to general formula (I) in a human subject, wherein said human subject is homozygous for ΔF508.

The present invention is equally applicable to other folding mutants of CFTR. Such other folding mutants may be characterized by a mutation of phenylalanine 508 or not. Subjects in which one allele (first allele) encodes a CFTR mutant characterized by a mutation of phenylalanine 508 of the CFTR protein and the other allele encoding a second CFTR mutant different from the one encoded by the first allele, but preferably also characterized by incorrect folding of the second CFTR mutant, are explicitly included in the patient subgroup according to preferred embodiments of the present invention.

In a second specific embodiment, said subject is characterized by at least one mutation in the CFTR gene which is causative for incorrect processing of the CFTR protein. Any mutation of this kind is also referred to herein as “processing mutation”, a term which is applicable both to the protein level and to the level of the nucleic acid that encodes the same. Preferably, in this second embodiment, the subject to be treated according to the present invention encodes a CFTR protein with a folding mutation (same or different) on each of the two alleles of the CFTR gene. When the folding mutation is identical on both alleles, which is preferred, then the subject is homozygous for said folding mutation.

The first and the second specific embodiments are not necessarily mutually exclusive. In other words, a subject eligible for treatment according to the present invention may be characterized both by a folding mutation or by a folding mutation, or by a mutation which is both a folding mutation and a processing mutation (i.e. causative for both incorrect processing and incorrect folding), on the same allele or on different alleles.

Although the CFTR mutation ΔF508 is categorized herein as “folding mutant”, the present invention should not be understood to be limited to such categorization, as it cannot be excluded that a re-categorization will be proposed in the scientific community; for example, some authors have also proposed the CFTR mutation ΔF508 to be categorized as “processing mutation”, see e.g. Cormet-Boyaka et al., 2004, Proc. Natl. Acad. Sci. USA, vol. 101, p. 8221-8226.

Preferably, the present invention is equally applicable to other processing mutants of CFTR. Such other folding mutants may be characterized by a mutation of phenylalanine 508 or not. Subjects in which one allele (first allele) encodes a CFTR mutant characterized by a mutation of phenylalanine 508 of the CFTR protein and the other allele encoding a second CFTR mutant different from the one encoded by the first allele, but preferably characterized by incorrect processing of the second CFTR mutant, are explicitly included in the patient subgroup according to preferred embodiments of the present invention.

Unless corrected, e.g. by administration of a suitable CFTR corrector, a processing mutation can be causative for a reduced presence of the CFTR protein in the cell, particularly reduced display of the CFTR protein at the cell surface, and/or for an altered molecular weight of the CFTR protein, compared to wild type CFTR protein. Display of the protein at the cell surface may be detectable e.g. by immunostaining. Presence of the protein and altered molecular weight may be detectable, for example, by gel electrophoresis and Western Blot.

In general, CFTR-processing mutants fail to leave the endoplasmic reticulum and are rapidly degraded. One example of a human CFTR processing mutant is characterized by the substitution of amino acid residue histidine 1085 by an arginine residue (H1085R, Cormet-Boyaka et al., 2004, Proc. Natl. Acad. Sci. USA, vol. 101, p. 8221-8226). Other processing mutants may be identified and/or have been described in the literature, and the present invention may be applicable to these as well.

Preferably, the at least one mutation is a mutation of the CFTR gene present in the cells of the respiratory tract of said subject. Without wishing to be bound to any particular theory, it is presently understood that any non-spontaneous mutation present in the germ line of a subject is normally also present in the respiratory tract of said subject. Presence of a mutation in the respiratory tract can be tested e.g. by taking a sample from the respiratory tract and gene sequence analysis, e.g. of the CFTR gene.

In some embodiments, said subject suffers from symptoms of cystic fibrosis in the respiratory tract. Symptoms of cystic fibrosis in the respiratory tract may include, without limitation, one or more of the following: clogging of the airways due to mucus build-up, decreased mucociliary clearance, and resulting inflammation), and difficulties in breathing. Without wishing to be bound to any particular theory, inflammation and infection cause injury and structural changes to the lungs, leading to a variety of symptoms. Further symptoms of cystic fibrosis in the respiratory tract can also include incessant coughing, copious phlegm production, and decreased ability to exercise. Without wishing to be bound to any particular theory, many of these symptoms occur when bacteria that normally inhabit the thick mucus grow out of control and cause pneumonia. Further symptoms of cystic fibrosis in the respiratory tract can also include changes in the architecture of the lung, such as pathology in the major airways (bronchiectasis), severe difficulties in breathing, coughing up blood (hemoptysis), high blood pressure in the lung (pulmonary hypertension), heart failure, difficulties getting enough oxygen to the body (hypoxia), and respiratory failure requiring support with breathing masks. In some embodiments, a subject suffering from symptoms of cystic fibrosis in the respiratory tract is infected by one or more of the following: Staphylococcus aureus, Haemophilus influenzae, and Pseudomonas aeruginosa; co-infection by other organisms is not excluded.

In some embodiments, said subject suffers from symptoms of cystic fibrosis in the gastrointestinal tract. Symptoms of cystic fibrosis in the gastrointestinal tract include, without limitation, thickened secretions from the pancreas, partial or complete blockage of the exocrine movement of pancreatic excretions into the duodenum and damage to the pancreas, often with painful inflammation (pancreatitis) and atrophy of the exocrine glands. The term “cystic fibrosis” refers to characteristic fibrosis and cysts that form within the pancreas (Andersen, 1938, Am. J. Dis. Child, vol. 56, p. 344-399), but is not generally limited to symptoms in the gastrointestinal tract. Indeed, in some embodiments, said subject suffers from symptoms of cystic fibrosis in the respiratory tract and also in the gastrointestinal tract.

In some embodiments, the subject to be subjected to therapy according to the present invention suffers from cystic fibrosis in the small airways. Small airways are usually defined as non-cartilaginous airways with an internal diameter <2 mm (Burgel et al., 2009, Eur. Respir. Rev., vol. 18, p. 80-95). Without wishing to be bound to a particular theory, small airways are often particularly vulnerable because many particles and infectious agents may be deposited there and because their narrow lumen makes them more susceptible to complete obstruction than larger airways. Normally, the epithelium of subjects suffering from cystic fibrosis in the small airways is affected by the disease, and respective subjects can profit from treatment by prevention or therapy, as described herein. Thus, the cystic fibrosis symptoms in the small airways epithelium may be prevented or treated according to the present invention. Thus, in preferred embodiments, the subject suffers from cystic fibrosis in small airway epithelium.

In the examples reported herein, the human CFBE41o- cell line was used as a model of cystic fibrosis (see Examples). As described previously, the CFBE41o- cell line is a human cell line that has been generated by transformation of cystic fibrosis (CF) tracheo-bronchial cells with SV40 and has been reported to be homozygous for the ΔF508 mutation (Ehrhard et al., 2006, Cell Tissue Res., vol. 323, p. 405-415). The CFBE41o- cell line is homozygous for ΔF508-CFTR over multiple passages in culture and expresses a number of proteins relevant for pulmonary absorption of pharmaceutical agents (e.g. P-gp, LRP and caveolin-1). This cell line retains at least some aspects of human CF bronchial epithelial cells, such as the ability to form electrically tight cell layers with functional cell-cell contacts, when grown under immersed (but not air-interfaced) culture conditions. Therefore, the CFBE41o- cell line is accepted as being useful for studies of cystic fibrosis, e.g. by treatment with small molecule agents (drug candidates) and for the gathering of further information about the disease at the cellular level, without the need for primary culture (Ehrhard et al., supra).

Description of the Compound For Use According to the Present Invention

The present invention relates to the treatment of cystic fibrosis in a subject belonging to a specific patient subgroup, as described herein, by a compound of general formula (I)

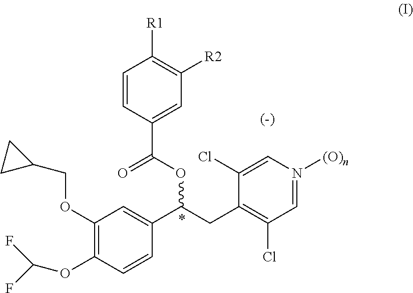

wherein:

n is 0 or 1;

R1 and R2 may be the same or different, and are selected from the group consisting of:
linear or branched C₁-C₆ alkyl, optionally substituted by one or more halogen atoms;
OR3 wherein R3 is a linear or branched C₁-C₆ alkyl optionally substituted with one or more halogen atoms or C₃-C₇ cycloalkyl groups; and
HNSO₂R4 wherein R4 is a linear or branched C₁-C₄ alkyl optionally substituted with one or more halogen atoms,
wherein at least one of R1 and R2 is HNSO₂R4, the pharmaceutically acceptable inorganic or organic salts, hydrates, solvates or addition complexes thereof.

The term “halogen atoms” as used herein includes fluorine, chlorine, bromine and iodine, preferably chlorine.

As used herein, the expression “linear or branched C1-Cx alkyl” where x is an integer greater than 1, refers to straight and branched chain alkyl groups wherein the number of carbon atoms is in the range 1 to x. Particular alkyl groups are methyl, ethyl, n-propyl, isopropyl and t-butyl. Optionally in said groups one or more hydrogen atoms can be replaced by halogen atoms, preferably chlorine or fluorine.

As used herein, the expression “C3-Cx cycloalkyl”, where x is an integer greater than 3, refers to cyclic non-aromatic hydrocarbon groups containing 3 to x ring carbon atoms. Examples include cyclopropyl, cyclobutyl, cyclopentyl, cyclohexyl and cycloheptyl. Optionally in said groups one or more hydrogen atoms can be replaced by halogen atoms, preferably chlorine or fluorine.

It will be apparent to those skilled in the art that compounds of general formula (I) contain one asymmetric center at the position of —CHO— and therefore exist as optical stereoisomers.

Although the present invention may comprise the use of a racemate or of the (−) or (+) enantiomers, preferably in substantially pure form, preferred compounds of formula (I) are (−) enantiomers. Example 2 shows that the (−) enantiomer of a compound according to general formula (I) has an effect as CFTR corrector. Thus, the compound according to general formula (I) is a substantially pure (−) enantiomer of a 1-phenyl-2-pyridinyl alkyl alcohol derivative.

Preferred groups of compounds of general formula (I) are those wherein:
R1 is HNSO₂R4, R2 is OR3 and n is 0;
R1 is HNSO₂R4, R2 is OR3 and n is 1;
R1 is HNSO₂R4, wherein R4 is methyl, R2 is OR3, wherein R3 is cyclopropylmethyl and n is 0;
R1 is HNSO₂R4, wherein R4 is methyl, R2 is OR3, wherein R3 is cyclopropylmethyl and n is 1;
R1 is linear or branched C₁-C₆ alkyl, R2 is HNSO₂R4 and n is 0;
R1 is methyl, R2 is HNSO₂R4, wherein R4 is methyl and n is 0;
R1 is linear or branched C₁-C₆ alkyl, R2 is HNSO₂R4 and n is 1;
R1 is methyl, R2 is HNSO₂R4, wherein R4 is methyl and n is 1;
R2 is linear or branched C₁-C₆ alkyl, R1 is HNSO₂R4 and n is 0;
R2 is methyl, R1 is HNSO₂R4, wherein R4 is methyl and n is 0;
R2 is linear or branched C₁-C₆ alkyl, R1 is HNSO₂R4 and n is 1;
R2 is methyl, R1 is HNSO₂R4, wherein R4 is methyl and n is 1;
R1 is OR3, R2 is HNSO₂R4 and n is 0;
R1 is OR3, R2 is HNSO₂R4 and n is 1;
R1 is OR3 wherein R3 is cyclopropylmethyl, R2 is HNSO₂R4 and R4 is methyl and n is 1;
R1 is OR3, R2 is HNSO₂R4 and n is 1;
both R1 and R2 are HNSO₂R4 and n is 0;
both R1 and R2 are HNSO₂R4, wherein R4 is methyl and n is 0;
both R1 and R2 are HNSO₂R4 and n is 1;
both R1 and R2 are HNSO₂R4, wherein R4 is methyl and n is 1.

Preferably, in the compound of general formula (I), R1 is HNSO₂R4; R4 is suitably methyl. Preferably, in the compound of general formula (I), R2 is OR3; R3 is suitably cyclopropylmethyl. Preferably, in the compound of general formula (I), n is 1.

In one preferred embodiment, the compound of formula (I) is a compound wherein R1 is HNSO₂R4, wherein R4 is methyl, R2 is OR3, wherein R3 is cyclopropylmethyl and n is 0.

In one preferred embodiment, the compound of formula (I) is a compound wherein R1 is OR3, R2 is HNSO₂R4, wherein R4 is methyl and n is 1—see Compound C2 in the Table 1 below.

In one preferred embodiment, the compound of formula (I) is a compound wherein R1 is methyl, R2 is HNSO₂R4 wherein R4 is methyl and n is 1.

In one preferred embodiment, the compound of formula (I) is a compound wherein both R1 and R2 are HNSO₂R4, wherein R4 is methyl and n is 0.

In one preferred embodiment, the compound of formula (I) is a compound wherein both R1 and R2 are HNSO₂R4, wherein R4 is methyl and n is 1.

Thus, according to these preferred embodiments, the present invention provides the use of the compounds reported in the table 1 below:

| Compound | Chemical name |
|---|---|
| C1 | 3-Cyclopropylmethoxy-4-methanesulfonylamino-benzoic acid 1-(3- |
| | cyclopropylmethoxy-4-difluoromethoxy-phenyl)-2-(3,5-dichloro-pyridin-4- |
| | yl)-ethyl ester |
| C2 | 3-Cyclopropylmethoxy-4-methanesulfonylamino-benzoic acid 1-(3- |
| | cyclopropylmethoxy-4-difluoromethoxy-phenyl)-2-(3,5-dichloro-1-oxy- |
| | pyridin-4-yl)-ethyl ester (CHF6001) |
| C3 | 4-Cyclopropylmethoxy-3-methanesulfonylamino-benzoic acid 1-(3- |
| | cyclopropylmethoxy-4-difluoromethoxy-phenyl)-2-(3,5-dichloro-1-oxy- |
| | pyridin-4-yl)-ethyl ester |
| C4 | 3,4-Bis-methanesulfonylamino-benzoic acid 1-(3-cyclopropyl-methoxy-4- |
| | difluoromethoxy-phenyl)-2-(3,5-dichloro-1-oxy-pyridin-4-yl)-ethyl ester |
| C5 | 3-Methanesulfonylamino-4-methyl-benzoic acid 1-(3-cyclopropyl-methoxy- |
| | 4-difluoromethoxy-phenyl)-2-(3,5-dichloro-1-oxy-pyridin-4-yl)-ethyl ester |
| C6 | 4-Methanesulfonylamino-3-methyl-benzoic acid 1-(3-cyclopropylmethoxy- |
| | 4-difluoromethoxy-phenyl)-2-(3,5-dichloro-1-oxy-pyridin-4-yl)-ethyl ester |

In some embodiments, compound C2 (CHF6001 is most preferred). CHF6001 was also used in the experimental examples shown herein. In the literature compound C2 has also been referred to under the name “[(S)-3,5-dichloro-4-(2-(3-(cyclopropylmethoxy)-4-(difluoromethoxy)phenyl)-2-(3-(cyclopropylmethoxy)-4-(methylsulfonamido)benzoyloxy)ethyl)pyridine 1-oxide] (CHF6001)” (Moretti et al. 2015 supra).

Thus, in line with the above, the patient subgroup to be specifically treated with the compound according to general formula (I) according to the present invention is represented by a subset of the total of subjects affected by cystic fibrosis.

It is generally known that the CFTR protein is activated by cAMP. While, based on this general knowledge, it had been previously proposed that some PDE4 inhibitors may promote activation of the CFTR protein, and thus of CFTR-dependent chloride secretion in certain respiratory diseases (Lambert et al., Am. J. Respir. Cell Mol. Biol., 2014, vol. 50, p. 549-558; Liu et al, J. Pharmacol Exp Ther., 2005, vol. 314, p. 846-854), it could never be shown that PDE4 inhibitors in general can promote activation of the CFTR protein. What is more, based on findings in the prior art, inhibition of PDE inhibitors alone cannot normally be expected to cure cystic fibrosis, in particular to correct the presence of the CFTR protein or its display at the cell surface (Blanchard et al., 2014, FASEB J., vol. 28, p. 791-801).

Notwithstanding the above, in some embodiments, the compound according to general formula (I) for use according to the present invention, indeed has PDE4 inhibitory activity. Without wishing to be bound to any particular theory, it is however envisaged that the PDE4 inhibition is not necessary and/or not sufficient for the mechanistic explanation of the effect of the compound of general formula (I) on the mutant CFTR protein in the patient subgroup to be treated, particularly on correction of CFTR. In particular, it is understood that PDE4 inhibitory activity cannot fully explain the observed correction of the presence of mutant CFTR encoded by a CFTR gene having at least one mutation, according to the present invention.

The findings of the present inventors are highly surprising in light of the prior art: when PDE4 inhibitors were previously tested and evaluated for their potential effect on wild-type and ΔF508-CFTR cells, it was found that PDE4 inhibitors alone produced minimal channel activation; they were found to amplify the effects of both CFTR correctors and CFTR potentiators, but nothing on CFTR correction by PDE4 inhibitors was suggested, let alone experimentally shown (Blanchard et al., 2014, FASEB J., vol. 28, p. 791-801).

Some PDE4 inhibitors, such as particularly roflumilast (Daxas, Takeda Pharmaceuticals, Zürich, Switzerland), are associated with adverse effects when administered to human subjects, in particular gastrointestinal disturbances such as nausea, diarrhea, abdominal pain, vomiting and dyspepsia (Moretto et al., 2015, J. Pharmacol. Exper. Ther., 2015, vol. 352, p. 559-567). While it was found, in the process of arriving at the present invention, that the known PDE4 inhibitor roflumilast may have a role in modulating some aspects of the ΔF508 CFTR protein, the present invention as specifically claimed is not centered on a use of roflumilast. In contrast to agents like roflumilast, the compound according to general formula (I) of the present invention is characterized by minimal adverse effects when administered to a subject. Thus, while cystic fibrosis may be treated or prevented in a subject by a compound according to the present invention, administration of such compound does not usually cause undesired adverse effects in most subjects. Adverse effects may occur e.g. when starting, continuing, increasing administration regimen or discontinuing a treatment. Sometimes adverse effects may cause complications of a disease or procedure and negatively affect its prognosis. They may also lead to non-compliance with a treatment regimen.

Example 1 demonstrates that some agents with known PDE4 inhibitory activity, in particular a compound of general formula (I) and roflumilast have a specific effect as CFTR potentiators. Among these, the compound of general formula (I) and roflumilast is associated with an advantageous adverse effect profile e.g. in human subjects.

This is a marked advantage over roflumilast, which, according to Example 1 could also be shown to correct the levels of ΔF508 CFTR, but which is well known to be associated with common adverse effects in subjects (e.g. in 1-10% of subjects), including diarrhea, weight loss, nausea, headache, insomnia, decreased appetite, abdominal pain, rhinitis, sinusitis, urinary tract infection and psychic disorders including depression (see e.g. Daliresp: EPAR—Product Information, European Medicines Agency, Takeda GmbH, 26 Sep. 2013).

Thereby, in the quest for an agent without off-target effects that normalizes mutant CFTR folding, processing, and function to resemble that of wild-type CFTR (Rowe et al., Cold Spring Harb. Perspect. Med., 2013, vol. 3, a009761), the provision of the specific use of the compound of the present invention in the treatment or prevention of cystic fibrosis in specific subjects is an important achievement.

It is well established that PDE4 exists in two distinct forms representing different conformations, that were designated as high affinity rolipram binding site or HPDE4, especially present in the central nervous system and in parietal cells, and low affinity rolipram binding site or LPDE4 (Jacobitz, et al, 1996, Mol. Pharmacol, vol. 50, p. 891-899), found in the immune and inflammatory cells. While both forms appear to exhibit catalytic activity, they differ with respect to their sensitivity to inhibitors. In particular compounds with higher affinity for LPDE4 appear less prone to induce side-effects such as nausea, emesis and increased gastric secretion. Therefore, in preferred embodiments, the compound for use according to the present invention is characterized by high PDE4 selectivity, in particular high LPDE4 selectivity. Indeed, it was shown in WO 2010/089107 A1, that compounds falling under general formula (I) of the present invention have excellent LPDE4 selectivity. Advantageously, the compounds of the invention are characterized by selectivity toward LPDE4 higher than that toward HPDE4 as obtained by the determination of their IC₅₀. According to the present invention, the IC₅₀ of a given agent with PDE4 inhibitory activity is to be determined as described in detail in WO 2010/089107 A1. Preferably, the HPDE4/LPDE4 IC₅₀ ratio for the compound for use according to the present invention is higher than 5, preferably higher than 10, more preferably higher than 20 and most preferably higher than 100.

The high PDE4 selectivity is a significant advantage over PDE4 inhibitors of the first generation such as rolipram and piclamilast, which are associated with strong adverse effects such as nausea and emesis and gastric acid secretion, and also an improvement in over second-generation PDE4 inhibitors such as cilomilast and roflumilast. As described in WO 2010/089107 A1, the presence of sulphonamido substituents on the benzoate residue in the compound of general formula (I) improves the potency, and the (−) enantiomer of the compound of general formula (I) is pharmaceutically advantageous over the corresponding (+) enantiomers and racemates.

In some embodiments, the compound to be used according to the present invention is a phosphodiesterase inhibitor which does not preferentially act as inhibitor of a cGMP-dependent phosphodiesterase. In some embodiments, the compound to be used according to the present invention does not specifically inhibit phosphodiesterase 5 (PDE5), i.e. is not a specific PDE5 inhibitor. Thereby, the compound of the present invention and its mode of action is different from the known phosphodiesterase 5 (PDE5) inhibitor Sildenafil, which had been previously investigated in an in vitro study for its potential dual function as CFTR corrector and potentiator (Leier et al., 2012, Cell Physiol. Biochem., vol. 29, p. 77-790); however the necessary high doses of the agent for CFTR recovery lead the authors to conclude that sildenafil might not be suited as therapeutic agent for treating cystic fibrosis lung disease. PDE5 is cGMP-dependent rather than cAMP-dependent.

In one embodiment, the present invention relates to the use of a compound according to general formula (I) as a CFTR corrector. In preferred embodiments, the present invention relates to the use of a compound according to general formula (I) as a corrector of a CFTR folding mutant and/or of a CFTR processing mutant.

In the most preferred embodiment, the compound to be used according to the present invention is 3-Cyclopropylmethoxy-4-methanesulfonylamino-benzoic acid 1-(3-cyclopropylmethoxy-4-difluoromethoxy-phenyl)-2-(3,5-dichloro-1-oxy-pyridin-4-y1)-ethyl ester. This corresponds to compound C2 in the Table above; the respective compound has been described previously (Armani et al., 2014, J. Med. Chem., vol. 57, p. 793-816; Moretto et al., 2015, J. Pharmacol. Exper. Ther., 2015, vol. 352, p. 559-567), but its direct action on mutant CFTR protein has so far not been proposed, let alone experimentally shown.

CHF 6001 is presently under development for the treatment of chronic obstructive pulmonary disease (COPD) and asthma. Good safety and tolerability of CHF 6001 in healthy volunteers has already been demonstrated at daily doses of up to 4800 μg for 14 days (Lucci et al., Eur. Resp. J., 2016, vol. 48, PA4086).

In one embodiment, the present invention relates to the use of CHF6001 as a CFTR corrector. In preferred embodiments, the present invention relates to the use of CHF6001 as a corrector of a CFTR folding mutant and/or of a CFTR processing mutant.

Preparation of Compounds Useful in the Present Invention

The compound is the compound of general formula (I) as defined herein. Such compounds useful in this invention and related compounds can be prepared by one of skill in the art by methods as disclosed in WO 2010/089107 A1, WO 2009/018909 A2, or by any other suitable method. In particular, the preparation of a compound according to general formula (I) may involve the synthesis of a racemic alcohol which is condensed with a chiral acid such as (S)-naproxen or (S)-acetylmandelic acid to obtain respectively a diastereomeric mixture, which is separated into two single diastereoisomers respectively e.g. by chromatography, crystallization or other well-known methods, giving after cleavage, respectively enantiomeric alcohols, that, by reaction with a suitable benzoic acid, give compounds of general formula (I), all as described in detail e.g. in WO 2010/089107 A1. Further aspects and examples are also described in WO 2010/089107 A1, and these are all applicable to the present invention.

Compositions comprising such compounds can also be prepared by methods as disclosed in WO 2010/089107 A1, WO 2009/018909 A2, or by any other suitable method.

Compositions

In one embodiment, the compound according to general formula (I) is substantially pure. “Substantially pure” as used herein means at least greater than about 97% is chirally pure, preferably greater than 99% and most preferably greater than 99.9%.

The compound according to general formula (I) for use according to the present invention may be formulated in a pharmaceutical composition. A pharmaceutical composition comprises a compound as described herein as useful in the present invention and at least one pharmaceutically acceptable salt, buffer substance, preservative, carrier, diluent and/or excipient. The term “pharmaceutically acceptable” describes something non-toxic and/or which does not substantially interact with the action of the active ingredient of the pharmaceutical composition.

The invention also encompasses the use of pharmaceutically acceptable hydrates, solvates, addition complexes, inorganic or organic salts of the compound according to general formula (I), e.g. sodium, potassium and lysine salts.

The pharmaceutical composition is preferably sterile and optionally comprises one or more further agents, mentioned or not mentioned herein.

Possible formulations include without limitation tablets, gelcaps, capsules, caplets, granules, lozenges and bulk powders; aqueous and non-aqueous solutions, emulsions, suspensions, syrups, and elixirs; creams, gels, pastes, foam, ointments, liniments, lotions, emulsions, suspensions, gels, pastes, powders, sprays, and drops; and transdermal patches. Inhalable preparations include inhalable powders, such as dry powders, propellant-containing metering aerosols or propellant-free inhalable formulations. Inhalable preparations are preferred in the present invention for the prevention or treatment of cystic fibrosis in the lungs.

Administration

In the present invention the compound of formula (I) is administered to a subject. In particular, all aspects and embodiments of the present invention foresee that the compound of formula (I) is administered to a subject in need thereof. A subject in need thereof is a subject characterized by at least one mutation in the CFTR gene which is causative for incorrect folding and/or processing of the CFTR protein, as described in detail throughout this specification.

Administration of the compound for use according to the present invention may be accomplished according to patient needs, for example, orally, nasally, parenterally, e.g. subcutaneously, intravenously, intramuscularly, intrasternally and by infusion, by inhalation, rectally, vaginally, topically, locally, transdermally, and by ocular administration.

For the treatment of cystic fibrosis of the respiratory tract, the compound for use according to the present invention is preferably administered by inhalation. In one embodiment, the compound of general formula (I) is administered by inhalation. One of the advantages of the inhalatory route over the systemic one is the possibility of delivering the agent directly at site of action, avoiding any systemic side-effects, thus resulting in a more rapid clinical response and a higher therapeutic ratio. The present invention in particular provides agents and pharmaceutical compositions for use by inhalation.

Indeed, the compounds for use according to the present invention, such as CHF6001 in particular, are optimal for inhaled delivery (Armani et al., 2014, J. Med. Chem., vol. 57, p. 793-816). The use by inhalation is particularly advantageous in the treatment of conditions of the respiratory tract, such as, in the present invention, cystic fibrosis in the respiratory tract.

The dosage of the compound for use according to the present invention depends upon a variety of factors including the particular condition to be treated or prevented, the severity of the symptoms, the route of administration, the frequency of the dosage interval, the particular compound utilized, the efficacy, toxicology profile, and pharmacokinetic profile of the compound. When the compound according to the present invention is administered to a subject by inhalation route, the dosage of the compound is advantageously comprised in the range of 0.01 to 20 mg/day, preferably between 0.1 to 10 mg/day, more preferably between about 0.5 to about 5 mg/day. Good safety and tolerability of CHF 6001 in healthy volunteers has already been demonstrated at daily doses of up to 4.8 mg for 14 days (Lucci et al., Eur. Resp. J., 2016, vol. 48, PA4086). In one embodiment, the compound of general formula (I) is administered once per day, but any alternative administration regime is also possible.

In one embodiment, the compound of general formula (I) is administered by a device selected from a single- or multi-dose dry powder inhaler, a metered dose inhaler and a soft mist nebulizer.

For administration of certain inhalable preparations, such as e.g. a dry powder, single- or multi-dose inhalers known from the prior art may be utilized. In that case the powder may be filled in gelatine, plastic or other capsules, cartridges or blister packs or in a reservoir. For that purpose, a diluent or carrier, generally non-toxic and chemically inert to the compounds of the invention, e.g. lactose or any other additive suitable for improving the respirable fraction may be added to powdered compounds of the invention.

For administration of further certain inhalable preparations, such as e.g. inhalation aerosols, containing propellant gas, such as hydrofluoroalkanes, may contain the compound for use according to the present invention either in solution or in dispersed form. Propellant-driven formulations may also contain other ingredients such as co-solvents, stabilizers and optionally other excipients. Propellant-free inhalable formulations comprising the compounds of the invention may be in form of solutions or suspensions in an aqueous, alcoholic or hydroalcoholic medium and they may be delivered by jet or ultrasonic nebulizers known from the prior art or by soft-mist nebulizers such as Respimat®.

Combinations

The compounds of the invention may be administered as the sole active agent or in combination with one or more other pharmaceutical active ingredients.

Thus, in a first embodiment, a compound according to general formula (I) is administered in a monotherapy. Monotherapy in this context means that additional therapeutic agents, i.e. additional pharmaceutically active components, other than the compound according to general formula (I), are not part of the treatment regimen foreseen according to the present invention. Indeed, the experimental examples of the present invention render plausible that a compound according to general formula (I) has on its own the desired therapeutic effect causative for the treatment of a subject which is characterized by at least one mutation in the CFTR gene which is causative for incorrect folding and/or processing of the CFTR protein. In that regard, the effect of the compounds according to general formula (I) of the present invention differ markedly from the compounds described e.g. by WO 2015/175773 A1 and Blanchard et al., 2014, FASEB J., vol. 28, p. 791-801.

In some embodiments, the compound of general formula (I) is used or administered in combination with at least one second pharmaceutically active component. At least one second pharmaceutically active component is preferably not a compound of general formula (I). Thus, the present invention also pertains to a combination therapy with at least two agents, wherein at least one agent is a compound according to general formula (Ii) and at least one agent is not a compound according to general formula (I). The at least two agents may be formulated together or separately.

Example 1 and Example 2 make plausible that the combined use of a compound according to general formula (I) with at least one further agent can provide a therapeutic benefit.

The at least one further agent is not particularly limited and includes small molecule agents as well as pharmaceutically active peptides or proteins and nucleic acids encoding the same, although certain agents are preferred, as specified in the following.

In one preferred embodiment, the at least one second pharmaceutically active compound is a CFTR corrector. Said CFTR corrector is not particularly limited and may be selected among all compounds and compositions which have the ability to act as CFTR correctors, as defined herein. Having said that, said CFTR corrector is preferably an agent which is not a compound according to general formula (I). In a preferred embodiment, said CFTR corrector is selected from the group comprising lumacaftor, VX-152 (Vertex Pharmaceuticals), VX-440 (Vertex Pharmaceuticals), VX-445 (Vertex Pharmaceuticals), tezacaftor (VX-661, Vertex Pharmaceuticals, see also Rowe et al., 2017, N. Engl. J. Med., vol. 377, p. 2024-2035), VX-659 (Vertex Pharmaceuticals), FDL 169 (Flatley Discovery Lab), GLPG2222 (Galapagos), PTI-801 (Proteostasis Therapeutics), and is preferably lumacaftor.

In a second preferred embodiment, the second pharmaceutically active compound is a CFTR potentiator. Said CFTR potentiator is not particularly limited and may be selected among all compounds and compositions which have the ability to act as CFTR potentiators, as defined herein. Said CFTR potentiator is preferably an agent which is not a compound according to general formula (I). In a preferred embodiment, said CFTR potentiator is selected from the group comprising ivacaftor, QWB251 (in development by Novartis), VX-561 (formerly CTP-656, Vertex Pharmaceuticals), PTI-808 (Proteostasis Therapeutics), genistein (De Stefano et al., 2014, Autophagy, vol. 10, p. 2053-2074), and is preferably ivacaftor.

In a third preferred embodiment, the second pharmaceutically active compound is a CFTR amplifier. Said CFTR amplifier is not particularly limited and may be selected among all compounds and compositions which have the ability to act as CFTR amplifiers, as defined herein. Said CFTR amplifier is preferably an agent which is not a compound according to general formula (I). In a preferred embodiment, said CFTR potentiator is selected from the group comprising PTI-CH (Molinski et al., 2017, EMBO Molecular Medicine, vol. 9, p. 1224-1243), PTI-428 (Proteostasis Therapeutics). It is known that amplifier compounds can provide an additional benefit for subjects affected by the ΔF508 mutation of CFTR, and the present invention provides the combined use of a compound of general formula (I) and a CFTR amplifier in these and other subjects.

In a further preferred embodiment, embodiment, the second pharmaceutically active compound is a compound capable of correcting the nucleotide sequence of mutant CFTR protein, either at DNA level (gene therapy) or at RNA level. A compound of the second class is QR-010 (ProQR Therapeutics).

In a further preferred embodiment, the second pharmaceutically active compound is a proteostasis regulator, preferably selected from cysteamine or a pharmaceutically acceptable salt thereof, such as preferably cysteamine bitrartrate (mercaptamine bitartrate, Cystagon®), and epigallocatechin gallate (EGCG), or a combination of two such proteostasis regulators (Tosco et al., Cell Death Differentiation, 2016, vol., 23, p. 1380-1393).

In a further embodiment, the second pharmaceutically active compound is selected from agents suitable to treat cystic fibrosis manifestations, preferably selected from the group of antibiotics, mucolytics, anti-inflammatory agents and aqueous salt solutions, particularly e.g. nebulized hypertonic saline.

In a particularly preferred embodiment, the at least one second pharmaceutically active compound comprises a combination of a CFTR corrector (other than the compound according to general formula (I)) and a CFTR potentiator (other than the compound according to general formula (I)); in other words, both a CFTR corrector and a CFTR potentiator can be foreseen for combination therapy together with the (other than the compound according to general formula (I)). Alternatively, a CFTR amplifier may be used for such combination therapy.

When the compound according to general formula (I) is formulated together with at least one further agent, then the compound according to general formula (I) and the at least one further agent are optionally present in the same composition. Thus, all compositions described herein may be formulated as compositions which contain, in addition to the agent according to general formula (I), at least one further agent, as specified herein. The preparation and administration of respective compositions is comprised in the present invention.

Alternatively, the compound according to general formula (I) and the at least one further agent are formulated in separate compositions. This may be appropriate e.g. when different routes of administration and/or different dosages are foreseen for the compound according to general formula (I) and the at least one further agent, respectively, and/or when the chemical properties and/or stability of the compound according to general formula (I) and the at least one further agent may require so. For example, in cases where it is foreseen to administer the compound according to general formula (I) by inhalation, but the at least one further agent by a route different from inhalation, separate formulations or compositions are appropriate. Having said that, the present invention explicitly also pertains to a kit of parts which comprises both, the compound according to general formula (I) and the at least one further agent, in separate formulations, but foreseen for combination therapy, at same or different time points.

In some embodiments, which are expressly combinable with all the above embodiments, at least one second pharmaceutically active compound is selected from one or more antibiotics, which may be given intravenously, inhaled, or by mouth, together with the compound according to general formula (I) or not.

### INDUSTRIAL APPLICABILITY

The present invention is of value for the treatment of cystic fibrosis patients. It is applicable to a variety of industries, including the chemical industry, pharmaceutical industry, other industries of the health sector, such as e.g. hospitals. It has also implications on related industries, e.g. insofar as packaging and labelling of drugs and/or diagnosis of patients (genotyping, phenotyping) are concerned.

### EXAMPLES

The following examples and figures are intended to illustrate some preferred embodiments of the invention and should not be interpreted to limit the scope of the invention, which is defined by the claims.

Material and Methods

Cell Lines

The CF human bronchial epithelial cell line (CFBE41o-), homozygous for the ΔF508 mutation of the CFTR protein (Ehrhard et al., 2006, Cell Tissue Res. Vol. 323, p. 405-415; kind gift from D. C. Gruenert, California Pacific Medical Center Research Institute, San Francisco, Calif., USA) and the human bronchial epithelial cell line 16HBE14o-, wildtype (wt) for the CFTR protein (kindly provided by P. Davis, Case Western Reserve University School of Medicine, Cleveland, Ohio, USA) were maintained in EMEM (Lonza) supplemented with 10% FBS and 1% Glutamax (Sigma). For polarized CFBE41o- monolayers, cells were seeded at a density of 2×10⁴/cm² onto Flask or multi-well pre-coated with a Fibronectin Coating Solution composed of LHC basal medium (Gibco, Invitrogen), 10% Bovine Serum Albumin (1 mg/ml), 1% of bovine Collagen I (Sigma) and Human Fibronectin (BD Laboratories) at final concentration 1 mg/ml, filtered (0.22 μM) before use. Cells are trypsinized with the PET™ dissociation reagent (at the effective date commercially available from different commercial suppliers, e.g. AthenaES), which contains polyvinylpyrrolidone, EGTA and trypsin in a HBS base.

Substances
CHF6001, MW 687.54
Roflumilast (CHF5152), MW 403.21
CHD-051662 (VX809, lumacaftor), MW 452.41
CHD-051663 (VX770, ivacaftor), MW 392.49

For CHF6001 and roflumilast, stock solutions were prepared by dissolving the compounds in DMSO at 5 mM. Stock solutions were incubated in an ultrasonic sonicator bath for 30 min and then kept for further 30 min at 37° C. For Lumacaftor and Ivacaftor, stock solutions were prepared by dissolving the compounds in DMSO at 5 mM. All stock solutions were maintained at −30° C. until use.

Flow Cytometry

CFBE41o- cells were seeded 1.5×10⁵ cells/well into a 6 multi-well dish in EMEM medium, supplemented with 10% FBS and 1mM L-glutamine, and maintained in incubator at 37° C. overnight. The day after, the cells were treated with different agents, as follows: VRT809 (5 μM), CHF6001 (30 nM), Roflumilast (50 nM) or the vehicle DMSO. After 24 h cells were harvested.

Specifically for detection of the extracellular domain of CFTR, the cells were washed with PBS 1× and successively stained with the anti-CFTR monoclonal antibody CF3 (Abcam), suitable for detection of the extracellular domain of CFTR. After washing, the secondary antibody goat anti-mouse (μ-chain) conjugated with Alexa Fluor-488 (Invitrogen, Carlsbad, U.S.A.) was added (1 μg for 10⁶ cells) for 30 min on ice.

Specifically, to recognize the c-terminal region of CFTR, after treatment with permeabilization wash buffer according to the manufacturer (BioLegend), cells were incubated (45 min at room temperature) with a polyclonal primary rabbit anti-CFTR antibody (Alomone Labs, Jerusalem, Israel). To decrease nonspecific binding, human serum (10% v/v) was added to the sample prior to its incubation with the primary antibody. To measure the contribution of nonspecific antibody-cell interactions, the rabbit polyclonal primary antibody was pre-incubated with a blocking peptide (4 mg) corresponding to amino acid residues 1,468-1,480 of CFTR, located in the C-terminal domain of CFTR. A Goat anti-rabbit IgG antibody (1.5 mg per sample) conjugated with Alexa Fluor (AF) 488 (Life Technologies, Carlsbad, Calif.) was used as secondary antibody.

Finally cells were washed twice and then analysed at MACSQuant Analyzer (Miltenyi Biotech, Cologne, Germany), and data were analysed with FlowJo Software (Tree Star, Inc).

The percentage of events with background noise (determined with IgM isotype or peptide signal) was subtracted and the result was expressed as %-values of CFTR positive cells. Geometrical means of the signal in the green channel were also obtained and a ratio between the signals obtained with the extracellular-domain specific antibody CF3 and with the polyclonal antibody (Alomone), respectively, with respect to the baseline signal was calculated (MFI).

To determine a possible cytotoxic activity of the above agents, the same agent-treated cells were also analysed using dual staining with fluorescent Annexin V and Propidium iodide (PI), in which Annexin V-positive/PI-negative cells are regarded as apoptotic cells and PI-positive cells as necrotic cells. After treatment cells were collected and incubated with 2.5 μl/ml Annexin V-eFluor 450/Binding Buffer 1× (eBioscience, Annexin V apoptosis detection kit eFluor 450) and left 10-15 min at room temperature. Then cells were incubated with 1 μl PI/300 μl of Binding Buffer and analysed by flow cytometry within 30 minutes, storing at 4° C. in the dark. The acquisition was performed using MACSQuant and data were analysed with FlowJo software.

HS-YFP Assay

The CFTR activity in epithelial cells was evaluated by Yellow Fluorescence Protein (YFP) with a protocol modified from the method published by Averna et al. (PLoS One, 2013, vol. 8, e66089). In accordance with Averna et al., the YFP was halide-sensitive, and the cells were not transfected with nucleic acid, but said YFP, purified from a recombinant source, was added to the supernatants to perform the assay; the modification with respect to Averna et al. specifically concerned the recombinant source of said YFP (here E. coli-expressed).

Western Blot

CFBE41o- and 16HBE14o- cells, respectively, were lysed in lysis buffer (50 mmol/L Tris, 150 mmol/L NaCl, 1 mmol/L EDTA, 1% Triton X-100 pH 7.4) containing protease inhibitors (Roche, Inc.). A total of 10 μg (16HBE14o-/CFBE41o-) total protein per lane was separated using 7.5% (v/v) polyacrylamide electrophoresis (PAGE) SDS gels and transferred onto nitro-cellulose membranes, that were probed with a monoclonal anti-CFTR antibody (Cell Signaling 2269; according to information provided by Cell Signaling produced by immunizing rabbits with a synthetic peptide corresponding to amino acid residues near the amino terminus of human CFTR) at a 1:500 dilution, overnight at 4° C. Membranes were re-probed with a monoclonal anti-actin (Sigma-Aldrich) to normalize for protein loading. The relative levels of CFTR were estimated by densitometry using the ImageJ program (http://rsb.info.nih.gov/ij/). The amount of band C (understood to be the fully glycosylated mature form of CFTR)) is calculated as a fraction of actin for the respective lane and reported as a fraction of the total (band C/actin). In general, band C in Western blot indicates that CFTR is correctly folded and has been processed in the Golgi apparatus. The values reported are expressed as means+/−SD (n=3). Data sets were compared by a t-test using GraphPad Prism.

Transepithelial Electrical Resistance (TEER) Assay

Transepithelial electrical resistance (TEER) is a widely accepted quantitative technique to measure the integrity of tight junction dynamics in cell culture models of endothelial and epithelial monolayers (Srinivasan et al., 2015, J. Lab. Autom., vol. 20, p. 107-126.) TEER values, indicated in Ohms, are good indicators of the integrity of the cellular barriers, e.g. prior to evaluation of agents on the cellular barrier. TEER measurements can be performed in real-time without cell damage. Thus, the experimentally determined TEER of a cellular monolayer is a quantitative measure of the barrier integrity and also a measure of its permeability to ions. The setup for measurement of TEER, as described herein, consists of a cellular monolayer cultured on a semipermeable filter insert (Costar Transwell®, Corning, USA, 12 mm insert, 0.4 μm Polyester Membrane) that defines a partition for apical and baso lateral compartments. The surfaces of insert were coated with the Fibronectin coating solution. For electrical measurements, two electrodes are used, with one electrode placed in the upper compartment and the other in the lower compartment, and the electrodes are separated by the cellular monolayer. The ohmic resistance is calculated based on Ohm's law as the ratio of the voltage and current; an alternating current (AC) voltage signal with a square waveform is applied. A TEER measurement system known as an Epithelial Voltohmmeter (EVOM; World Precision Instruments, Sarasota, Fla.) was used, applied an AC square wave at a frequency of 12.5 Hz. An EVOM and its use is illustrated in FIG. 7. The EVOM system has a measurement range of 1-9999Ω with a 1Ω resolution, and it uses a pair of electrodes known as a STX2/“chopstick” electrode pair. Each stick of the electrode pair (4 mm wide and 1 mm thick) contains a silver/silver chloride pellet for measuring voltage and a silver electrode for passing current. The measurement procedure includes measuring the blank resistance (R_{BLANK}) of the semipermeable membrane only (without cells) and measuring the resistance across the cell layer on the semipermeable membrane (R_{TOTAL}). The cell-specific resistance (R_{TISSUE}), in units of Ω, can be obtained as:

R_{TISSUE}(Ω)=R_{TOTAL}−R_{BLANK}

TEER values are reported in units of Ω*cm² and calculated as:

TEER_{REPORTED}=R_{TISSUE}(Ω)*M_{AREA}(cm²)

For the TEER assay, cells were grown for 3-7 days prior to experiments and medium was changed two times a week. Some of the cells were exposed to agents as follows: either the inhibitor, CFTRᵢₙₕ-172 (40 μM), or activators of CFTR: IBMX (100 μM) and Forskolin (10 μM), VRT 770 (5 μM), or the inhibitor of ENaC Amiloride (200 μM) or one of the following agents: CHF6001 (30 nM) or Roflumilast (50 nM). Controls were exposed to DMSO (1:1000). The agents were added to the medium apically and basally, and the TEER was measured after 10, 30 and 60 minutes.

Immunofluorescence

CFBE41o- and 16HBE14o- cells, respectively, were seeded on a glass slide and, after exposure to agent or vehicle, were washed twice with PBS 1× and fixed with paraformaldehyde 4% (PFA) for 30 min and stored in PBS 1× at 4° C. until immunostaining. The fixed cells were washed twice with PBS 1×, and treated for 3 minutes with 50 nM NH₄Cl at room temperature in order to quench the aldehyde group. After another washing step, the cells were permeabilized with TRITON X100 0.1% for 5 minutes and were blocked with a solution of 1% BSA for 30 minutes. The anti-CFTR antibody M3A7, raised against an epitope corresponding to residues 1197-1480 of human CFTR (Santa Cruz) was added at 1:100 dilution for 1 hour, then the cells were stained with secondary antibody anti IgG1 488 (1:1000, Santa Cruz) and Rhodamine Phalloidine (1:500) for another hour. Finally, the cells were subjected to DAPI (Sigma Aldrich) staining (1:2000) for 1 hour at RT, then the slides were analyzed by Leica DM6000M microscope with a 40× objective. Images were processed for brightness and contrast with Adobe Photoshop.

Statistical Analysis

Statistical analyses were performed by Prism5 software (GraphPad Software Inc., La Jolla, U.S.A.) A one-way ANOVA was used to compare means of variables between groups. All pair-wise comparisons were performed using the Tukey's post-hoc test. A significance threshold of p b 0.05 was set for all statistical analyses.

### Example 1: CFTR Potentiator Activity of CHF6001

The potential effect of different agents on the activity of CFTR in CFBE41o- cells was analysed by the HS-YFP assay, combining a short exposure (10 minutes) with agents as follows: CHF6001, Roflumilast or VRT 770 (5 μM), alone or in combination, with a 24 h pre-treatment with the CFTR corrector VRT809 (5 μM). For concentrations of the agents used see FIG. 1.

The results are shown in FIG. 1. These results demonstrate a significant ability of both agents to stimulate CFTR activity.

Of note is the observation that, following CFTR correction by the CFTR corrector VRT809, CHF6001 restored the CFTR activity in CFBE41o- cells to levels comparable to the reference compound VRT 770.

By the TEER assay, shown in FIG. 2, the functionality of the apical channels present in the cells is determined by measuring the ion flux through the epithelium at different time points. The resistance decreases in function of the increase in the number of ions that pass the membrane through the channels in the unit of time. In order to verify the ability of CHF6001 and Roflumilast to act directly on CFTR channel, the TEER assay was performed with these agents on 16HBE14o- bronchial epithelial cells (BEC) grown in liquid-liquid interface.

As shown in FIG. 2A, the effect of different agent(s) was tested on 16HBE14o- cells treated for 10, 30, and 60 min, respectively, with CFTR-inhibitors, or Amiloride (a Na⁺ channel inhibitor). As expected, an increase of epithelial resistance was recorded, due to a reduced ion flux through epithelium. On the contrary, the CFTR potentiator Ivacaftor (VRT 770) and IBMX plus Forskolin produced a significant decrease in trans-endothelial electrical resistance estimated to be 60-70% as compared to control.

As shown in FIG. 2B, the agents CHF6001 (30 nM) and Roflumilast (50 nM) were tested on 16HBE14o- cells at different time-points. Values of trans-epithelial electric resistance (Ohm/cm²) were normalized to DMSO values (set to 100%). Measures were performed at 10, 30 and 60 minutes after exposure to the respective agent(s). Ivacaftor (VRT 770), CHF6001 (CHF) and Roflumilast (ROFL) appeared able to decrease the electric resistance in all experimental conditions tested. These data confirm the data obtained by HS-YFP assay and support a role for CHF6001, and also Roflumilast, as CFTR potentiators.

In summary, this example confirms that a compound of general formula (I) (CHF6001) has potentiator activity on ΔF508 CFTR.

### Example 2: CFTR Corrector Activity of CHF6001

The potential corrector activity of roflumilast and a compound of general formula (I) (CHF6001) was evaluated in comparison with the known CFTR corrector VRT809 in the human bronchial epithelial cell line CFBE41o- (homozygous for the ΔF508 mutation of the CFTR protein) by the HS-YFP assay (FIG. 3). For concentrations of the agents used see FIG. 3. Surprisingly, both roflumilast and CHF6001 induced CFTR activity to a level equal or superior to VRT809.

It was tested whether recovery of CFTR activity in the human bronchial epithelial cell line CFBE41o- (homozygous for the ΔF508 mutation of the CFTR protein) by roflumilast and CHF6001, respectively, could be associated with a recovery of the presence of CFTR at the cell surface. The known CFTR corrector VRT809 was used for comparison purposes. For concentrations of the agents used see FIG. 4. The presence of CFTR was evaluated by flow cytometry using two different antibodies that target extracellular (CF3) and the intracellular (Alomone) epitopes of CFTR. Interestingly, both CHF6001 and roflumilast were found to be capable to restore the presence of CFTR epitopes on CFBE41o- cells after 24 h of treatment. Notably, the observed restoration is comparable to the one observed with the reference compound VX809 (see FIG. 4).

Total presence of CFTR protein was also tested by exposing the human bronchial epithelial cell line CFBE41o- (homozygous for the ΔF508 mutation of the CFTR protein) to roflumilast, VRT809 and CHF6001 (for concentrations: see FIG. 5), respectively, followed by cell lysis, gel electrophoresis and Western Blot. Western blotting analysis of cell lysates and quantification of the total signal intensity, in percentage, confirming up-regulation of CFTR protein is shown in FIG. 5.

In summary, this example together with Example 1, confirms that a compound of general formula (I) (CHF6001) has both potentiator and corrector activities in cells characterized by the genotype CFTR F508del^{+/+} (i.e. mutation ΔF508 on both alleles of the CFTR gene).

### BRIEF DESCRIPTION OF THE FIGURES

FIG. 1: CFTR potentiator activity evaluated in CFBE41o- cells (characterized by the genotype CFTR F508del^{+/+} (i.e. mutation ΔF508 on both alleles of the CFTR gene)) and expressed as delta fluorescence between un-stimulated versus stimulated cells after short exposure (10 minutes) to CHF6001, Roflumilast or VRT770 at different concentrations alone or following preincubation (24 h) with VRT809.

Values are means±SEM (n=4). ** p≤0.02 *p≤0.05 t-test (DMSO vs treatment)

FIG. 2: A: TEER was performed after exposure of 16HBE14o- bronchial epithelial cells to CFTR-inhibitor (CFTR-inh 172, 40 μM), forskolin (10 μM)+IBMX(100 μM), amiloride (200 μM) or VRT 770 (Ivacaftor, 5 μM).

B: CHF6001 (30 nM), Roflumilast (50 nM) or VRT770 were tested on 16HBE14o- cells at different time-points. Values of trans-epithelial electric resistance (Ohm/cm²) of 16HBE14o- were normalized to DMSO values (set to 100%). Measures were performed at 10, 30 and 60 minutes after exposure to the agent(s).

Values are means±SEM (n=3). ** p≤0.02 * p≤0.05 t-test (DMSO vs treatment)

FIG. 3. CFTR corrector activity was evaluated in CFBE41o- epithelial cell line after 24 h exposure to CFTR corrector VRT809 (5 μM), CHF6001, and Roflumilast at the indicated doses.

Values are normalized for total cell content and expressed as means±SEM (n=4) *p≤0.05 t-test (vs DMSO)

FIG. 4. CFTR presence in human bronchial epithelial cell lines 16HBE14o- (non CF) and CFBE41o- (ΔF508/ΔF508) was evaluated by flow cytometry after 24 h exposure to CFTR corrector VRT809 (5 μM) in comparison with the two compounds CHF6001 (30 nM) or Roflumilast (50 nM). Flow cytometry was performed, following trypsin-mediated detachment of the cells from the culture dish, using two different antibodies that target extracellular (CF3) and intracellular (Alomone) epitopes of CFTR. The percentage of CFTR positive cells was already subtracted of events determinate by IgM isotype or peptide signal. Values are means±SEM (n=4). ** p≤0.02 *p≤0.05 t-test (DMSO vs treatment/16HBE14o- as non-CF reference). For example the bar on the very left of FIG. 4 shows that about 30% of cells were found positive for antibody staining.

FIG. 5. Presence of CFTR was evaluated by western blotting in human bronchial epithelial cell lines 16HBE14o- (non CF) and CFBE41o- (ΔF508/ΔF508) after 24 h exposure to CFTR corrector VRT809 (5 μM) in comparison with the two compounds CHF6001 and Roflumilast.

Top: average of several experiments; the relative levels of CFTR were estimated by densitometry using the ImageJ program (http://rsb.info.nih.gov/ij/). The native amount of band C is calculated as a fraction of actin for the respective lane and reported as a fraction of the total (band C/actin). The values reported are expressed as means+/−SEM (n=3). Data sets were compared by a t-test using GraphPad Prism. *p≤0.05 t-test (vs DMSO). % of CFTR presence is set to 100 in 16HBE14o- cells (i.e. non-CF cells).

Bottom: Western Blot of one representative experiment.

FIG. 6: Immunofluorescence staining performed on CFBE41o- cells treated for 24 h with VX809+VX770, CHF6001 and Roflumilast at different concentrations or vehicle (DMSO). Both PDE4 inhibitors increase the presence of CFTR (one representative of n=3 experiments).

FIG. 7: Voltohmmeter as used in the examples described herein

FIG. 8: amino acid sequence of the human CFTR protein (1480 amino acids). Accession P13569, version P13569.3, dbsource UniProtKB: locus CFTR_HUMAN

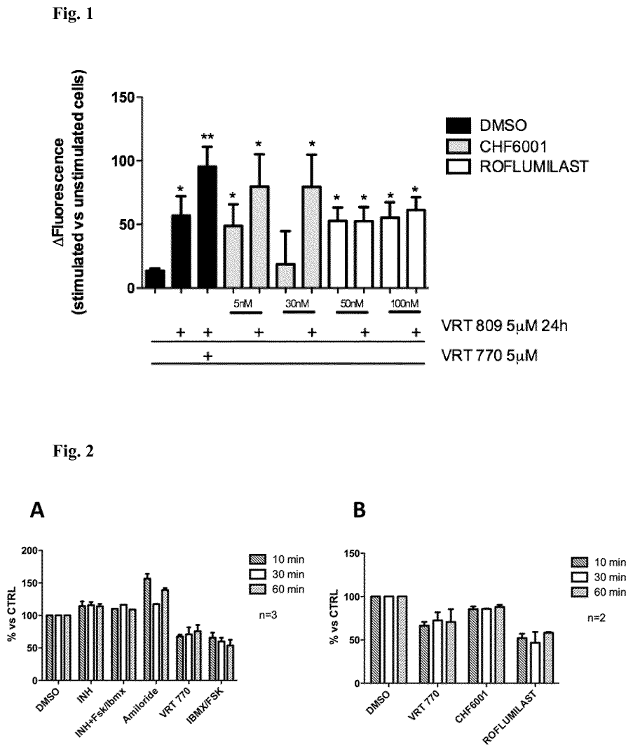

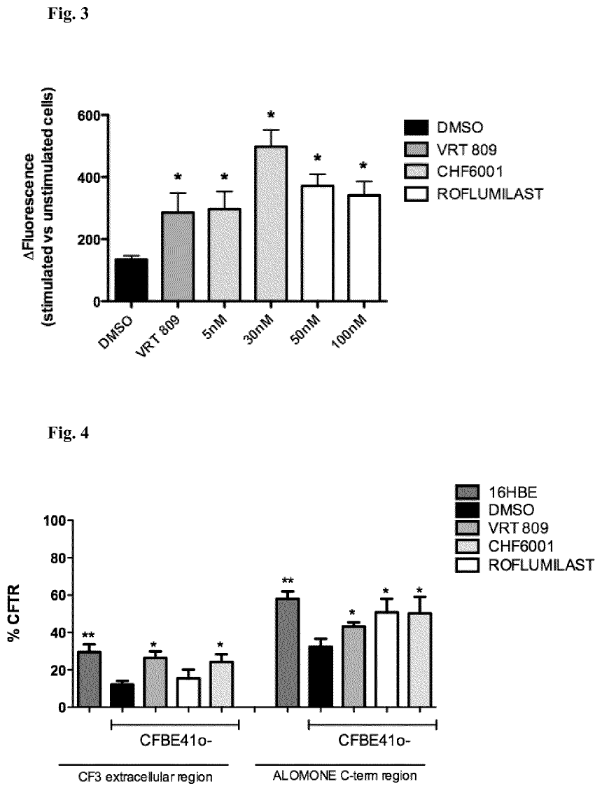

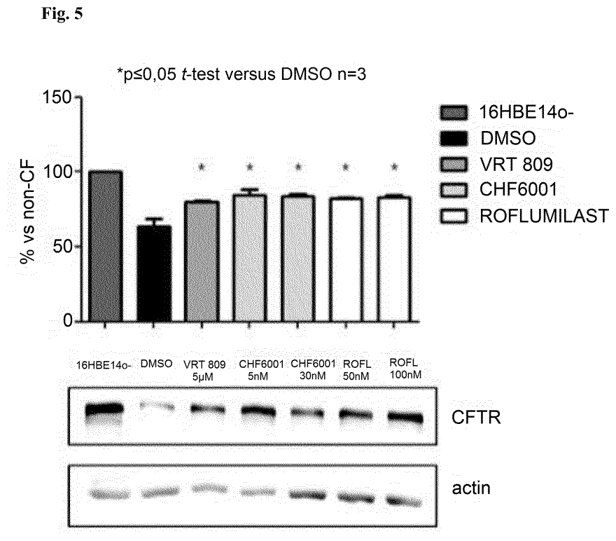

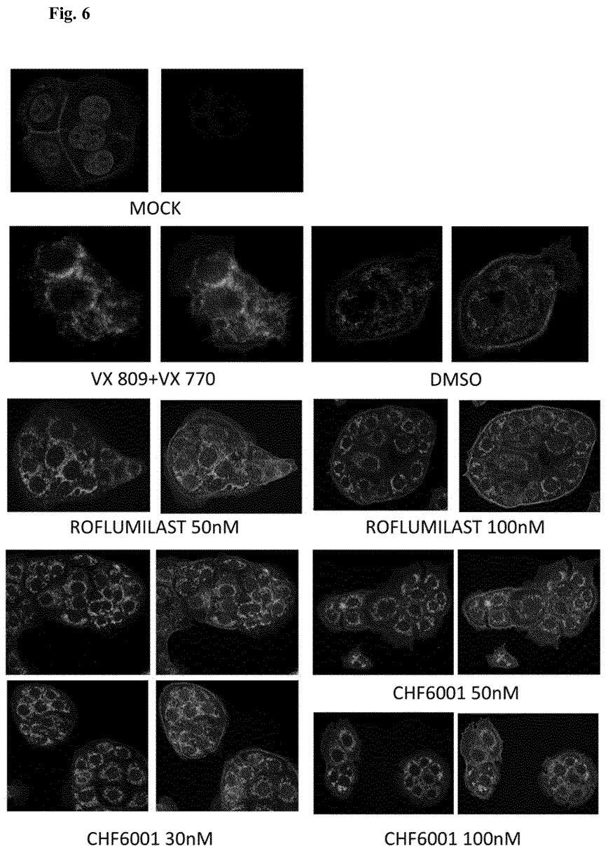

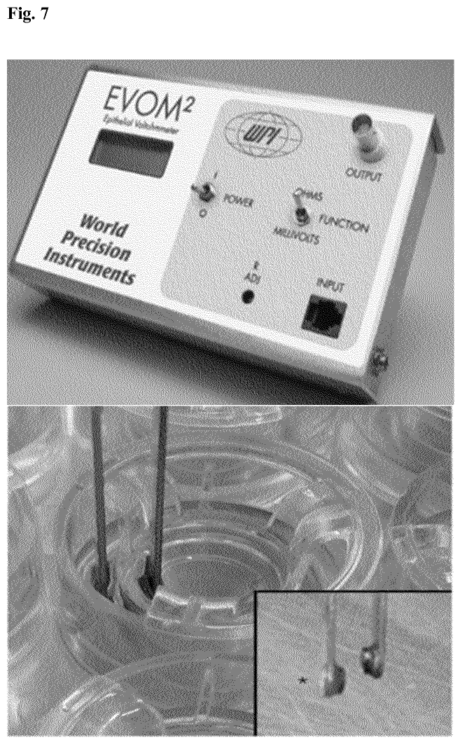

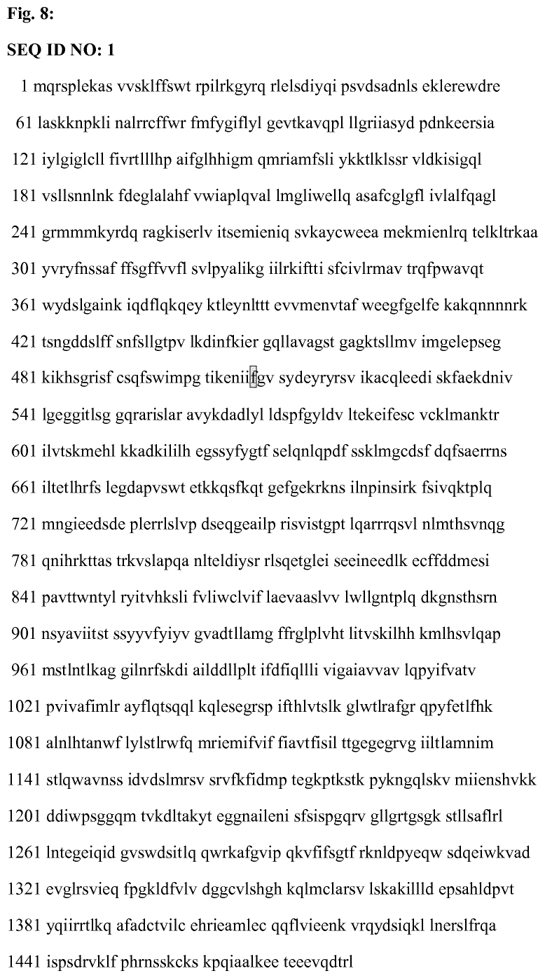

Phenylalanine 508 is highlighted.

1. A method for the prevention and/or treatment of cystic fibrosis, said method comprising administering to a subject in need thereof an effective amount of a compound of formula (I) as (−) enantiomer 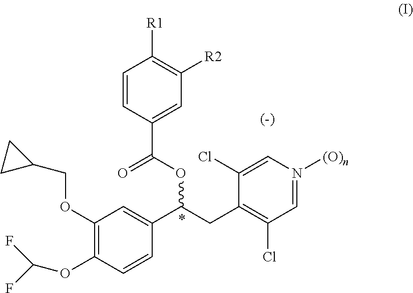
wherein:
n is 0 or 1;
R1 and R2 may be the same or different, and are selected from the group consisting of:
linear or branched C₁-C₆ alkyl, optionally substituted by one or more halogen atoms;
OR3 wherein R3 is a linear or branched C₁-C₆ alkyl optionally substituted with one or more halogen atoms or C₃-C₇ cycloalkyl groups; and
HNSO₂R4 wherein R4 is a linear or branched C₁-C₄ alkyl optionally substituted with one or more halogen atoms,
wherein at least one of R1 and R2 is HNSO₂R4,
a pharmaceutically acceptable inorganic or organic salt thereof, a hydrate thereof, a solvate thereof, or an addition complex thereof,
wherein said subject is characterized by at least one mutation in the CFTR gene which is causative for incorrect folding and/or processing of the CFTR protein.

2. The method according to claim 1, wherein R1 is HNSO₂R4, wherein R4 is methyl, R2 is OR3, wherein R3 is cyclopropylmethyl and n is 1.

3. The method according to claim 1, wherein the compound is selected from the group consisting of:
a. a compound wherein R1 is HNSO₂R4, wherein R4 is methyl, R2 is OR3, wherein R3 is cyclopropylmethyl and n is 0;
b. a compound wherein R1 is OR3, R2 is HNSO₂R4, wherein R4 is methyl and n is 1,
c. a compound wherein R1 is methyl, R2 is HNSO₂R4 wherein R4 is methyl and n is 1;
d. a compound wherein both R1 and R2 are HNSO₂R4, wherein R4 is methyl and n is 0; and
e. a compound wherein both R1 and R2 are HNSO₂R4, wherein R4 is methyl and n is 1.

4. The method according to claim 1, wherein said subject is characterized by at least one mutation in the CFTR gene which is causative for incorrect folding of the CFTR protein.

5. The method according to claim 1, wherein the compound has CFTR corrector activity.

6. The method according to claim 1, wherein said subject is characterized by at least one mutation in the CFTR gene which is causative for incorrect processing of the CFTR protein.

7. The method according to claim 6, wherein said at least one mutation is a genomic mutation of the CFTR gene and/or a mutation of the CFTR gene present in the cells of the respiratory tract of said subject.

8. The method according to claim 1, wherein the compound additionally has PDE4 inhibitory activity.

9. The method according to claim 1, wherein said subject is human.

10. The method according to claim 9, wherein the genome of said human subject encodes at least the mutation ΔF508 in the CFTR protein.

11. The method according to claim 9 or claim 10, wherein said human subject encodes the mutation ΔF508 in both genomic alleles of the gene encoding the CFTR protein (i.e. the subject is homozygous for ΔF508).

12. The method according to claim 1, wherein said subject suffers from symptoms of cystic fibrosis in the respiratory tract, in the gastrointestinal tract, or both.

13. The method according to claim 1, wherein the use said compound is administered by inhalation.

14. The method according to claim 13, wherein said compound is administered by a device selected from a single- or multi-dose dry powder inhaler, a metered dose inhaler and a soft mist nebulizer.

15. The method according to claim 1, wherein said compound is administered in combination with at least one second pharmaceutically active component selected from the group consisting of a CFTR corrector, a CFTR potentiator, and combinations thereof.

16. The method according to claim 15, wherein said second pharmaceutically active component is selected from the group consisting of ivacaftor and lumacaftor.


SEQUENCE LISTING (1 sequence)

SEQ ID NO: 1 - protein, 1480 aa, Homo sapiens
MQRSPLEKASVVSKLFFSWTRPILRKGYRQRLELSDIYQIPSVDSADNLSEKLEREWDRELASKKNPKLINALRRCFFWRFMFYGIFLYLGEVTKAVQPLLLGRIIASYDPDNKEERSIAIYLGIGLCLLFIVRTLLLHPAIFGLHHIGMQMRIAMFSLIYKKTLKLSSRVLDKISIGQLVSLLSNNLNKFDEGLALAHFVWIAPLQVALLMGLIWELLQASAFCGLGFLIVLALFQAGLGRMMMKYRDQRAGKISERLVITSEMIENIQSVKAYCWEEAMEKMIENLRQTELKLTRKAAYVRYFNSSAFFFSGFFVVFLSVLPYALIKGIILRKIFTTISFCIVLRMAVTRQFPWAVQTWYDSLGAINKIQDFLQKQEYKTLEYNLTTTEVVMENVTAFWEEGFGELFEKAKQNNNNRKTSNGDDSLFFSNFSLLGTPVLKDINFKIERGQLLAVAGSTGAGKTSLLMVIMGELEPSEGKIKHSGRISFCSQFSWIMPGTIKENIIFGVSYDEYRYRSVIKACQLEEDISKFAEKDNIVLGEGGITLSGGQRARISLARAVYKDADLYLLDSPFGYLDVLTEKEIFESCVCKLMANKTRILVTSKMEHLKKADKILILHEGSSYFYGTFSELQNLQPDFSSKLMGCDSFDQFSAERRNSILTETLHRFSLEGDAPVSWTETKKQSFKQTGEFGEKRKNSILNPINSIRKFSIVQKTPLQMNGIEEDSDEPLERRLSLVPDSEQGEAILPRISVISTGPTLQARRRQSVLNLMTHSVNQGQNIHRKTTASTRKVSLAPQANLTELDIYSRRLSQETGLEISEEINEEDLKECFFDDMESIPAVTTWNTYLRYITVHKSLIFVLIWCLVIFLAEVAASLVVLWLLGNTPLQDKGNSTHSRNNSYAVIITSTSSYYVFYIYVGVADTLLAMGFFRGLPLVHTLITVSKILHHKMLHSVLQAPMSTLNTLKAGGILNRFSKDIAILDDLLPLTIFDFIQLLLIVIGAIAVVAVLQPYIFVATVPVIVAFIMLRAYFLQTSQQLKQLESEGRSPIFTHLVTSLKGLWTLRAFGRQPYFETLFHKALNLHTANWFLYLSTLRWFQMRIEMIFVIFFIAVTFISILTTGEGEGRVGIILTLAMNIMSTLQWAVNSSIDVDSLMRSVSRVFKFIDMPTEGKPTKSTKPYKNGQLSKVMIIENSHVKKDDIWPSGGQMTVKDLTAKYTEGGNAILENISFSISPGQRVGLLGRTGSGKSTLLSAFLRLLNTEGEIQIDGVSWDSITLQQWRKAFGVIPQKVFIFSGTFRKNLDPYEQWSDQEIWKVADEVGLRSVIEQFPGKLDFVLVDGGCVLSHGHKQLMCLARSVLSKAKILLLDEPSAHLDPVTYQIIRRTLKQAFADCTVILCEHRIEAMLECQQFLVIEENKVRQYDSIQKLLNERSLFRQAISPSDRVKLFPHRNSSKCKSKPQIAALKEETEEEVQDTRL